(19) United States
(12) Patent Application Publication
(10) Pub. No.: US 2015/0202153 A1
(43) Pub. Date: Jul. 23, 2015

(54) LIPOSOMAL DRUG DELIVERY SYSTEM

(71) Applicant: UNIVERSITY OF THE WITWATERSRAND, JOHANNESBURG, Johannesburg (ZA)

(72) Inventors: Derusha Frank, Johannesburg (ZA); Viness Pillay, Sandton (ZA); Lisa Claire du Toit, Johannesburg (ZA); Yahya Essop Choonara, Johannesburg (ZA)

(21) Appl. No.: 14/433,285

(22) PCT Filed: Oct. 4, 2013
(86) PCT No.: PCT/IB2013/059116

§ 371 Date: Apr. 2, 2015

(30) Foreign Application Priority Data
Oct. 4, 2012 (ZA) 2012/07435

(51) Int. Cl. A61K 9/127; A61K 31/357; A61K 31/4745

(52) U.S. Cl. CPC A61K 9/1271; A61K 31/4745; A61K 31/357

(57) ABSTRACT

The invention relates to a drug delivery system, particularly to a liposomal drug delivery system. Most particularly the invention relates to a liposomal drug delivery system for the release of at least one drug compound at a target site within a human or animal body. The invention extends to a method of manufacturing the drug delivery system.

### FIELD OF THE INVENTION

The invention relates to a drug delivery system (DDS), particularly to a liposomal drug delivery system (LDDS). Most particularly, the invention relates to a liposomal drug delivery system (LDDS) for the release of at least one drug compound at a target site within a human or animal body, the liposomal drug delivery system (LDDS) comprising a liposomal shell consisting of distearoyl phosphocholine (DSPC) and either distearoyl phosphatidylethanolamine-m-PEG (DSPE-m-PEG) or cholesterol (CHO); and a drug compound housed inside the liposomal shell. The invention extends to a method of manufacturing the drug delivery system.

### BACKGROUND TO THE INVENTION

Cancer remains one of the most debilitating morbidities and a significant cause of mortality the world over, with clinical patterns highlighting a disturbing regression in the age of onset (Tang et al., 2009). Ovarian cancer is the most aggressive of all gynaecological cancers with a high incidence of recurrence and a deplorable five-year survival rate (Chien et al., 2007; Ferrandina et al., 2006). The poor prognosis of ovarian cancer can be attributed to the absence of overt symptoms and a lack of early detection mechanisms, resulting in advanced disease and metastasis at the time of diagnosis (Rose et al., 1996; Hornung et al., 1999; Ferrandina et al., 2006; Cirstoiu-Hapca et al., 2010; Kim et al., 2011).

Whilst cancer research is an extensive and dynamic field, an adequately safe and efficacious treatment modality still eludes the medical fraternity (Liu et al., 2009). Current treatment protocols for ovarian cancer involve surgical debridement as well as adjuvant chemotherapy with a taxane/platinum compound combination (Stuart, 2003; Cirstoiu-Hapca et al., 2010; Kim et al., 2011). Anti-neoplastic drugs as a class act indiscriminately on actively dividing tissue causing severe, often life-threatening, side effects including: immunosuppression, gastrointestinal disturbance, alopecia, cardiac complications and neuropathies (Vauthier et al., 2003; Cho et al., 2007; Cirstoiu-Hapca et al., 2010; Mohanty and Sahoo, 2010; Guo et al., 2011; Shapira et al., 2011). Furthermore, penetration of systemically administered chemotherapeutic agents into tumour tissue, and hence anti-tumour efficacy, is compromised by factors such as heterogeneity of tumoural vasculature, and high interstitial pressure within the tumour (Park J W, 2002). Therefore, the balance of achieving optimal anti-neoplastic therapy while minimising side-effects remains a very delicate one.

In addition to the non-specific biodistribution and consequent detrimental side effects, anti-neoplastic drugs also pose significant formulation challenges due to inherent poor aqueous solubility (Pathak et al., 2006). The intravenous (IV) route of administration offers substantial benefits in terms of efficacy of anti-neoplastic therapy as a consequence of enhanced bioavailability, whilst being minimally invasive. IV formulations of anti-neoplastic drugs often involve the utilisation of additional solubilizers and/or carrier vehicles, and/or complex formulation processes, each of which present their own shortfalls such as side-effects and increased production costs. It is for the above-mentioned reasons that clinical use of the model drug, camptothecin (CPT), was significantly reduced. Although extremely potent against a wide range of solid tumours, including ovarian cancer, the poor aqueous solubility of CPT coupled with a severe side-effect profile was a major drawback to the clinical usefulness of CPT (Schluep et al., 2006; Liu et al., 2009; Fan et al., 2010). Water-soluble derivatives of CPT, such as irinotecan and topotecan, have since been developed and used clinically. However, the efficacy of these derivatives is significantly lower than that of CPT.

The immense allure of nanostructures is a consequence of their unique properties, which distinguishes them from the bulk material from which they are derived (Knopp et al., 2009, Ranjan et al., 2009). Nanosystems for biomedical application are currently a highly researched field and have exhibited immense potential, particularly in the diagnostic, imaging and therapeutic domains (Dominguez and Lustegarten, 2010). Numerous benefits of nanosystems are related to the augmented surface area-to-volume ratio (Khosravi-Darani et al., 2007; Chen et al., 2011; Vizirianakis, 2011).

Liposomal drug delivery systems (LDDS) are known, and have many drawbacks. These drawbacks include the limited life-span of the liposomes in vivo and drug leakage from within the liposomes during storage (Madrigal-Carballo et al., 2010; Chun et al., 2013). Known LDDSs are often large, over about 200 nm in diameter, hindering accumulation at a target site.

There exists a need for drug delivery systems that can deliver anti-neoplastic drug compounds to turmour sites, preferably DDSs that can deliver lipophilic anti-neoplastic drug compounds bio-specifically to solid tumour sites, via intravenous (IV) administration such that the drug delivery system effectively targets the tumour and concentrates at the tumour site to effectively release the drug compound. There exists a need for drug delivery systems having the aforementioned qualities that are simple to manufacture, readily formulated into IV formulations, and are stable to allow for prolonged storage.

### SUMMARY OF THE INVENTION

In broad terms, the invention relates to a liposomal drug delivery system (LDDS) for the release of at least one drug compound at a target site in a human or animal body, the liposomal drug delivery system (LDDS) comprising a liposomal shell consisting of at least one phospholipid, the shell defining therein an inner compartment. The LDDS may further comprise a drug compound housed inside the inner compartment defined by the liposomal shell. The LDDS may further comprise a surfactant.

In accordance with a first aspect of this invention there is provided a liposomal drug delivery system (LDDS) for the release of at least one drug compound at a target site in a human or animal body, the liposomal drug delivery system (LDDS) comprising a liposomal shell consisting of distearoyl phosphocholine (DSPC) and distearoyl phosphatidylethanolamine-m-polyethylene glycol (DSPE-m-PEG), the shell defining therein an inner compartment.

The liposomal drug delivery system (LDDS) may further comprise a drug compound housed inside the inner compartment defined by the liposomal shell.

The distearoyl phosphocholine (DSPC) may be 1,2-distearoyl-sn-glycero-3-phosphocholine. The distearoyl phosphatidylethanolamine-m-polyethylene glyclol (DSPE-m-PEG) may be L-α-distearoylphosphatidylethanolamine-methoxy-polyethylene glycol conjugate.

The liposomal shell may further comprise a surfactant. The surfactant may be is at least one surfactant selected from the group consisting of, but not limited to: dioctyl sulfosuccinate (DOS), Tween 80 and Span 80, or any combination thereof, preferably the surfactant is dioctyl sulfosuccinate (DOS). The surfactant may in use increase the structural stability of the liposomal shell, and may facilitate formation of liposomal shells having dimensions that are nanosized.

The liposomal shell may be configured such that non-polar functional groups of the distearoyl phosphocholine (DSPC) and the distearoyl phosphatidylethanolamine-m-polyethylene glycol (DSPE-m-PEG) are directed inwardly toward the compartment and polar functional groups are directed outwardly toward an outer surface of the shell. In use, the non-polar functional groups of the liposomal shell increases the solubilisation of non-polar and/or lipophilic drug compounds such as camptothecin housed within the compartment.

The drug compound may be at least one drug compound selected from the group consisting of, but not limited to: amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, antiparkinson agents, antidepressants, anti-neoplastic agents, immunosuppressants, antiviral agents, antibiotic agents, antifungal agents, antimicrobial agents, appetite suppressants, antiemetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds, oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol-lowering agents, phytochemicals, and antioxidants, or any derivative of the aforementioned. The drug classes mentioned above are listed for illustrative purposes, the liposomal drug delivery system (LDDS) according to the invention may include any pharmaceutical formulation regardless of the active substance and/or substances incorporated therein.

Preferably the drug is at least one anti-neoplastic drug selected from the group consisting of, but not limited to: camptothecin, taxanes and platinum compounds, preferably the anti-neoplastic drug is camptothecin. In embodiments wherein the drug is camptothecin, the compartment provides protection for the housed drug from lactone ring opening typically taking place at physiological conditions in use. The non-polar groups of the liposomal shell facilitates housing non-polar drugs such as camptothecin (CPT), therein preventing drug leakage from the liposomal shell prior to the liposomal shell reaching the target site, in use.

The target site may be cancerous cells located in or on the human or animal body, preferably cancerous cells that are formed into a tumour, further preferably the tumour being an ovarian tumour.

The liposomal shell may have a diameter of less than about 200 nm, preferably less than about 160 nm. The liposomal shell may be sized so as to form a nanoliposome (NLS). In use, nanoliposomes increase the enhanced permeability and retention (EPR) effect, therein facilitating increased drug delivery to the target site. Liposomal shells having a diameter of about 200 nm or less, preferably less than about 160 nm, will facilitate successful targeting of the liposomal shells to the tumour.

The nanoliposome (NLS) may further contain a gas housed within the inner compartment defined by the shell so as to form a nanolipobubble (NLB) and thus a nano-lipobubble liposomal drug delivery system (NLB-LDDS).

The gas may be at least one gas selected from the group consisting of, but not limited to: air, nitrogen, oxygen, carbon dioxide, hydrogen, nitrous oxide, a noble or inert gas such as helium, argon, xenon or krypton; a radioactive gas such as Xe¹³³ or Kr⁸¹; a hyperpolarized noble gas, a low molecular weight hydrocarbon such as methane, ethane, propane, butane, isobutane, pentane or isopentane; a cycloalkane such as cyclobutane or cyclopentane; an alkene such as propene, butene or isobutene; or an alkyne such as acetylene; an ether; a ketone; an ester; halogenated gases, preferably fluorinated or perfluorinated gases, such as fluorinated hydrocarbons; sulphur hexafluoride; perfluoroacetone; perfluorodiethyl ether; perfluoroalkanes; perfluoroalkenes; perfluoroalkynes; perfluorocycloalkanes; and saturated perfluorocarbons. Preferably, the gas is sulphur-hexa fluoride.

In use, diffusion of the gas from the compartment out to the target site causes cavitation of the nanolipobubble (NLB) compromising its structural integrity and, in turn, facilitating release of the drug compound from within the compartment to the target site.

The liposomal shell may further comprise a polymeric coating at least partially covering the shell. The polymeric coating may be pH responsive so as to undergo a conformational change and compromise the structural integrity of the coating at pH values lower than physiological pH, more preferably at pH values similar to that of a cancerous tumour, typically about pH 6. The polymeric coating may be at least one polymeric coating selected from the group consisting of, but not limited to: biocompatible polymers, ionic polymers preferably anionic and/or cationic polymers. The ionic polymers may include but are not limited to: gelatin, polyethyleneimine (PEI), poly-L-lysine (PLL), carrageenan, pectin, sodium alginate, carboxylic polymers, sulfate, and amine functionalized polymers such as polyacrylic acid (PAA), polymethacrylic acid, polyethylene amine, polysaccharides such as alginic acid, pectinic acid, carboxy methyl cellulose, hyaluronic acid, heparin (mucopolysaccharide), chitosan, carboxymethyl chitosan, carboxymethyl starch, carboxymethyl dextran, heparin sulfate, chondroitin sulfate, cationic guar, cationic starch, and their salts, poly (butyl cyanoacrylate) (PBCA), poly(lactic acid) (PLA), poly(propylene fumarate)(PPF), polyanhydrides.

In a preferred embodiment of the invention the polymeric coating is a cationic polymer, further preferably chitosan.

In another embodiment of the invention the liposomal shell is coated with two or more coatings being sequentially layered. The two or more coatings which are sequentially layered preferably alternate between a cationic polymer coating and an anionic polymer coating. The cationic polymer coating is preferably chitosan (CHT), and the anionic polymer coating is preferably polyacrylic acid (PAA).

The liposomal shell may further comprise a lyoprotectant. Preferably, the lyoprotectant may be a sugar. The sugar may be at least one sugar selected from the group consisting of, but not limited to: lactose and fructose.

In a preferred embodiment of the first aspect of the invention there is provided for a nanoliposomal drug delivery system comprising:
a nanoliposomal shell consisting of distearoyl phosphocholine (DSPC), distearoyl phosphatidylethanolamine-m-polyethylene glycol (DSPE-m-PEG) and a surfactant, the shell defining an inner compartment; and
a drug compound housed inside the inner compartment defined by the nanoliposomal shell.

The nanoliposomal shell may further comprise a gas housed within the inner compartment so as to form a nanolipobubble (NLB).

The nanoliposomal shell and/or the nanolipobubble may further comprise a polymeric coating at least partially covering the shell.

According to a second aspect of this invention there is provided a liposomal drug delivery system (LDDS) for the release of at least one drug compound at a target site in a human or animal body, the liposomal drug delivery system comprising a liposomal shell consisting of distearoyl phosphocholine (DSPC) and cholesterol (CHO), the shell defining an inner compartment.

The distearoyl phosphocholine (DSPC) may be 1,2-distearoyl-sn-glycero-3-phosphocholine.

The liposomal drug delivery system may further comprise a drug compound housed inside the inner compartment defined by the liposomal shell.

The liposomal shell may further comprise a surfactant. The surfactant may be is at least one surfactant selected from the group consisting of, but not limited to: dioctyl sulfosuccinate (DOS), Tween 80 and Span 80, or any combination thereof, preferably the surfactant is dioctyl sulfosuccinate (DOS). The surfactant may in use increase the structural stability of the liposomal shell, and may facilitate formation of liposomal shells having dimensions that are nanosized.

The liposomal shell may be configured such that non-polar functional groups of the distearoyl phosphocholine (DSPC) and the cholesterol (CHO) are directed inwardly toward the compartment and polar functional groups directed outwardly toward an outer surface of the shell. In use, the non-polar functional groups of the liposomal shell increases the solubilisation of non-polar and/or lipophilic drug compounds such as camptothecin (CPT) housed within the compartment.

The drug compound may be at least one drug compound selected from the group consisting of, but not limited to: amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, antiparkinson agents, antidepressants, anti-neoplastic agents, immunosuppressants, antiviral agents, antibiotic agents, antifungal agents, antimicrobial agents, appetite suppressants, antiemetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds; oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol-lowering agents, phytochemicals, and antioxidants, or any derivative of the aforementioned. The drug classes mentioned above are listed for illustrative purposes, the liposomal drug delivery system (LDDS) according to the invention may include any pharmaceutical formulation regardless of the active substance and/or substances incorporated therein.

Preferably the drug is at least one anti-neoplastic drug selected from the group consisting of, but not limited to: camptothecin, taxanes and platinum compounds, preferably the anti-neoplastic drug is camptothecin. In embodiments wherein the drug is camptothecin, the compartment provides protection for the housed drug from lactone ring opening typically taking place at physiological conditions in use. The non-polar groups of the liposomal shell facilitates housing non-polar drugs such as camptothecin (CPT), therein preventing drug leakage from the liposomal shell prior to the liposomal shell reaching the target site, in use.

The target site may be cancerous cells, preferably cancerous cells formed into a tumour, further preferably the tumour being an ovarian tumour.

The liposomal shell may have a diameter of less than about 200 nm, preferably less than about 160 nm. The liposomal shell may be sized so as to form a nanoliposome (NLS). In use, nanoliposomes increase the enhanced permeability and retention (EPR) effect, therein facilitating increased drug delivery to the target site. Liposomal shells having a diameter of about 200 nm, preferably less than about 160 nm, will facilitate successful targeting of the liposomal shells to the tumour.

The nanoliposome (NLS) may further contain a gas housed within the inner compartment defined by the shell so as to form a nanolipobubble (NLB) and thus a nano-lipobubble liposomal drug delivery system (NLB-LDDS).

The gas may be at least one gas selected from the group consisting of, but not limited to: air, nitrogen, oxygen, carbon dioxide, hydrogen, nitrous oxide, a noble or inert gas such as helium, argon, xenon or krypton; a radioactive gas such as Xe¹³³ or Kr⁸¹; a hyperpolarized noble gas, a low molecular weight hydrocarbon such as methane, ethane, propane, butane, isobutane, pentane or isopentane; a cycloalkane such as cyclobutane or cyclopentane; an alkene such as propene, butene or isobutene; or an alkyne such as acetylene; an ether; a ketone; an ester; halogenated gases, preferably fluorinated or perfluorinated gases, such as fluorinated hydrocarbons; sulphur hexafluoride; perfluoroacetone; perfluorodiethyl ether; perfluoroalkanes; perfluoroalkenes; perfluoroalkynes; perfluorocycloalkanes; and saturated perfluorocarbons. Preferably, the gas is sulphur-hexa fluoride.

In use, diffusion of the gas from the compartment out to the target site causes cavitation of the nanolipobubble (NLB) compromising its structural integrity and, in turn, facilitating release of the drug compound from within the compartment to the target site.

The liposomal shell may further comprise a polymeric coating at least partially covering the shell. The polymeric coating may be pH responsive so as to undergo a conformational change and compromise the structural integrity of the coating at pH values lower than physiological pH, more preferably at pH values similar to that of a cancerous tumour, typically about pH 6. The polymeric coating may be at least one polymeric coating selected from the group consisting of, but not limited to: biocompatible polymers, ionic polymers preferably anionic and/or cationic polymers. The ionic polymers may include but are not limited to: gelatin, polyethyleneimine (PEI), poly-L-lysine (PLL), carrageenan, pectin, sodium alginate, carboxylic polymers, sulfate, and amine functionalized polymers such as polyacrylic acid (PAA), polymethacrylic acid, polyethylene amine, polysaccharides such as alginic acid, pectinic acid, carboxy methyl cellulose, hyaluronic acid, heparin (mucopolysaccharide), chitosan, carboxymethyl chitosan, carboxymethyl starch, carboxymethyl dextran, heparin sulfate, chondroitin sulfate, cationic guar, cationic starch, and their salts, poly (butyl cyanoacrylate) (PBCA), poly(lactic acid) (PLA), poly(propylene fumarate)(PPF), polyanhydrides.

In a preferred embodiment of the invention the polymeric coating is a cationic polymer, further preferably chitosan.

In another embodiment of the invention the liposomal shell is coated with two or more coatings being sequentially layered. The two or more coatings which are sequentially layered preferably alternate between a cationic polymer coating and an anionic polymer coating. The cationic polymer coating is preferably chitosan, and the anionic polymer coating is preferably polyacrylic acid.

The liposomal shell may further comprise a lyoprotectant. Preferably, the lyoprotectant may be a sugar. The sugar may be at least one sugar selected from the group consisting of, but not limited to: lactose and fructose.

In a preferred embodiment of the second aspect of the invention there is provided for a nanoliposomal drug delivery system comprising:
a liposomal shell consisting of distearoyl phosphocholine (DSPC), cholesterol (CHO), and a surfactant, the shell defining an inner compartment; and
a drug compound housed inside the inner compartment defined by the liposomal shell.

The nanoliposomal shell may further comprise a gas housed within the inner compartment so as to form a nanolipobubble (NLB).

The nanoliposomal shell and/or the nanolipobubble may further comprise a polymeric coating at least partially covering the shell.

According to a third aspect of this invention there is provided for use of a liposomal shell for the delivery of a drug compound to a target site in a human or animal body in the diagnosis and/or treatment of a disease, the liposomal shell consisting of distearoyl phosphocholine (DSPC) and distearoyl phosphatidylethanolamine-m-polyethylene glycol (DSPE-m-PEG), the shell defining an inner compartment.

The liposomal shell may further comprise a drug compound housed inside the inner compartment.

The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a fourth aspect of this invention there is provided for use of a liposomal shell in the manufacture of a medicament for the delivery of a drug compound to a target site in a human or animal body in the treatment of a disease, the liposomal shell consisting of distearoyl phosphocholine (DSPC) and distearoyl phosphatidylethanolamine-m-polyethylene glycol (DSPE-m-PEG), the shell defining an inner compartment.

The medicament may be formulated as an intravenous (IV) formulation.

The liposomal shell may further comprise a drug compound housed inside the inner compartment.

The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a fifth aspect of this invention there is provided for use of a liposomal shell for the delivery of a drug compound to a target site in a human or animal body in the treatment and/or diagnosis of a disease, the liposomal shell consisting of distearoyl phosphocholine (DSPC) and cholesterol (CHO).

The liposomal shell may further comprise a drug compound housed inside the inner compartment.

The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a sixth aspect of this invention there is provided for use of a liposomal shell in the manufacture of a medicament for the delivery of a drug compound to a target site in a human or animal body in the treatment of a disease, the liposomal shell consisting of distearoyl phosphocholine (DSPC) and cholesterol (CHO).

The medicament may be formulated as an intravenous (IV) formulation.

The liposomal shell may further comprise a drug compound housed inside the inner compartment.

The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a seventh aspect of this invention there is provided for a method of treating cancer, preferably ovarian cancer, by administering to a human or animal in need of cancer treatment a liposomal drug delivery system (LDDS) in accordance with a first and/or second aspect of this invention.

According to an eighth aspect of this invention there is provided a method of manufacturing the liposomal drug delivery system (LDDS) according to the first aspect of this invention, the method comprising the steps of:
adding DSPC and DSPE-m-PEG to an organic solvent, preferably a mixture of chloroform and methanol, to produce Solution 1;
adding a surfactant, preferably DOS, to Solution 1 to form Solution 2;
adding a drug compound, preferably CPT, to Solution 2 to form Solution 3;
adding phosphate buffered saline (PBS) to Solution 3 to form Solution 4; and
evaporating Solution 4 under vacuum to produce an aqueous solution of the LLDS.

According to a ninth aspect of this invention there is provided a method of manufacturing the liposomal drug delivery system (LDDS) according to the second aspect of this invention, the method comprising the steps of:
adding DSPC and cholesterol (CHO) to an organic solvent, preferably a mixture of chloroform and methanol, to produce Solution 1;
adding a surfactant, preferably DOS, to Solution 1 to form Solution 2;
adding a drug compound, preferably CPT, to Solution 2 to form Solution 3;
adding phosphate buffered saline (PBS) to Solution 3 to form Solution 4; and
evaporating Solution 4 under vacuum to produce an aqueous solution of the LLDS.

### BRIEF DESCRIPTION OF THE DRAWINGS

Embodiments of the invention will be described below by way of example only and with reference to the accompanying drawings in which:

FIG. 1 shows typical Zeta Size profiles (Size vs Intensity) of a DSPC:CHO nano-liposomal drug delivery system at T=0 hours (a) and T=3 hours (b) in accordance with a second aspect of this invention. FIG. 1 also shows typical Zeta Size profiles (Size vs Intensity) of a DSPC:DSPE-m-PEG nano-liposomal drug delivery system at T=0 (c) and T=3 (d) in accordance with a first aspect of this invention;

FIG. 2a-c shows fractional drug release for nano-liposomal drug delivery systems (LDDSs) in accordance with a first aspect of this invention with varying DSPC:DSPE-m-PEG ratios. FIG. 2d-f shows fractional drug release for nano-liposomal drug delivery systems (LDDSs) in accordance with a second aspect of this invention with varying DSPC:CHO (3:1-1:3) ratios;

FIG. 3 shows transmission electron photomicrographs of DSPC:CHO nano-liposomes (NLS) at 30000× magnification (a), 40000× magnification (b) and 50000× magnification (c), respectively;

FIG. 4 shows micro-ultrasound images of a) carrageenan hydrogel prior to introduction of DSPC:CHO nano-liposomes, b) injection of nano-liposomes and c) dispersion of nano-liposomes through the hydrogel 2 minutes post injection;

FIG. 5 shows size-intensity profiles of a) candidate CHO-NLS, b) CHO-NLB, c) candidate DSPE-m-PEG-NLS and d) DSPE-m-PEG-NLB. (in all cases n=3 and SD<0.02);

FIG. 6 shows scanning electron micrographs of post-lyophilization products of CHO-NLS coated with CHT and PAA (4800× magnification);

FIG. 7 shows fluorescence micrographs of a) CHO-NLB and b) DSPE-m-PEG-NLB labelled with FITC dye confirming the restoration of NLB structure following lyophilization, reconstitution and SF₆ gas introduction;

FIG. 8 shows graphical illustration of the post-modification DIEs of CPT and SB in CHO-NLB and DSPE-m-PEG-NLB;

FIG. 9 shows fractional drug release profiles of CPT from a) candidate CHO-NLS and DSPE-m-PEG-NLS and b) candidate CHO-NLB and DSPE-m-PEG-NLB, at tumoural and physiologic pH over 24 hours (in all cases n=3 and SD<0.02);

FIG. 10 shows fractional drug release of a) CPT and b) SB, from CHO-NLB and DSPE-m-PEG-NLB containing SB, at tumoural and physiologic pH over 24 hours (in all cases n=3 and SD<0.02);

FIG. 11 shows fractional drug release of a) CPT and b) SB, from layer-by-later CHT and PAA coated CHO-NLB and DSPE-m-PEG-NLB, at tumoural and physiologic pH over 24 hours (in all cases n=3 and SD<0.02);

FIG. 12 shows backscatter profiles of a) uncoated CHO-NLB and b) layer-by-layer CHT and PAA polymer coated CHO-NLB in non-reference mode, up to 12 hours post reconstitution at ambient temperature. Change in backscatter as a function of time of c) uncoated CHO-NLB and d) layer-by-layer CHT and PAA polymer coated CHO-NLB, with reference to the initial measurement;

FIG. 13 shows backscatter profiles of a) uncoated DSPE-m-PEG-NLB and b) layer-by-layer CHT and PAA polymer coated DSPE-m-PEG-NLB in non-reference mode, up to 12 hours post reconstitution at ambient temperature. Change in backscatter as a function of time of c) uncoated DSPE-m-PEG-NLB and d) layer-by-layer CHT and PAA polymer coated DSPE-m-PEG-NLB, with reference to the initial measurement; and

FIG. 14 shows the a) average size, b) zeta potential, c) CPT incorporation and D) SB incorporation of layer-by-layer CHT and PAA polymer coated CHO-NLB and DSPE-m-PEG-NLB stored under ambient and refrigeration temperatures over a three month period (in all cases n=3 and SD<0.03).

### DETAILED DESCRIPTION OF AN EMBODIMENT OF THE INVENTION

In broad terms, the invention relates to a liposomal drug delivery system (LDDS) for the release of at least one drug compound at a target site in a human or animal body, the liposomal drug delivery system (LDDS) comprising a liposomal shell consisting of at least one phospholipid, the shell defining therein an inner compartment. The LDDS may further comprise a drug compound housed inside the inner compartment defined by the liposomal shell. The LDDS may further comprise a surfactant.

In accordance with a first aspect of this invention there is provided a liposomal drug delivery system (LDDS) for the release of at least one drug compound at a target site in a human or animal body, the liposomal drug delivery system (LDDS) comprising a liposomal shell consisting of distearoyl phosphocholine (DSPC) and distearoyl phosphatidylethanolamine-m-polyethylene glycol (DSPE-m-PEG), the shell defining an inner compartment. Typically, the distearoyl phosphocholine (DSPC) is 1,2-distearoyl-sn-glycero-3-phosphocholine, and the distearoyl phosphatidylethanolamine-m-polyethylene glyclol (DSPE-m-PEG) is L-α-distearoylphosphatidylethanolamine-methoxy-polyethylene glycol conjugate (DSPE-m-PEG).

The liposomal drug delivery system (LDDS) typically comprises a drug compound housed inside the inner compartment defined by the liposomal shell. The drug compound may be at least one drug compound selected from the group consisting of, but not limited to amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, antiparkinson agents, antidepressants, anti-neoplastic agents, immunosuppressants, antiviral agents, antibiotic agents, antifungal agents, antimicrobial agents, appetite suppressants, antiemetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds, oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol-lowering agents, phytochemicals, and antioxidants, or any derivative of the aforementioned. The drug classes mentioned above are listed for illustrative purposes, the liposomal drug delivery system (LDDS) according to the invention may include any pharmaceutical formulation regardless of the active substance and/or substances incorporated therein.

Preferably the drug is at least one anti-neoplastic drug selected from the group consisting of, but not limited to: camptothecin, taxanes and platinum compounds, preferably the anti-neoplastic drug is camptothecin. In embodiments wherein the drug is camptothecin, the compartment provides protection for the housed drug from lactone ring opening typically taking place at physiological conditions in use. The non-polar groups of the liposomal shell facilitates housing non-polar drugs such as camptothecin (CPT), therein preventing drug leakage from the liposomal shell prior to the liposomal shell reaching the target site, in use.

The liposomal shell may further comprise a surfactant. The surfactant may be is at least one surfactant selected from the group consisting of, but not limited to: dioctyl sulfosuccinate (DOS), Tween 80 and Span 80, or any combination thereof, preferably the surfactant is dioctyl sulfosuccinate (DOS). The surfactant may in use increase the stability of the liposomal shell. The surfactant is typically adsorbed into or onto the liposomal shell. The higher the concentration of the surfactant in the liposomal shell the better the stabilization and the smaller the liposomal shells formed. The surfactant facilitates manufacturing liposomal shells having nano dimensions.

The liposomal shell is typically configured such that non-polar functional groups of the distearoyl phosphocholine (DSPC) and the distearoyl phosphatidylethanolamine-m-polyethylene glycol (DSPE-m-PEG) are directed inwardly toward the compartment and polar functional groups are directed outwardly toward an outer surface of the shell. In use, the non-polar functional groups of the liposomal shell increases the solubilisation of non-polar and/or lipophilic drug compounds such as camptothecin housed within the compartment.

The target site is typically cancerous cells located in or on the human or animal body, preferably cancerous cells that are formed into a tumour, further preferably the tumour being an ovarian tumour.

The liposomal shell may have a diameter of less than about 200 nm, preferably less than about 160 nm. The liposomal shell may be sized so as to form a nanoliposome (NLS). In use, nanoliposomes increase the enhanced permeability and retention (EPR) effect, therein facilitating increased drug delivery to the target site. Liposomal shells having a diameter of about 200 nm, preferably less than about 160 nm, will facilitate successful targeting of the liposomal shells to the tumour.

The nanoliposome (NLS) typically further contains a gas housed within the inner compartment defined by the shell so as to form a nanolipobubble (NLB) and thus a nano-lipobubble liposomal drug delivery system (NLB-LDDS). The gas may be at least one gas selected from the group consisting of, but not limited to: air, nitrogen, oxygen, carbon dioxide, hydrogen, nitrous oxide, a noble or inert gas such as helium, argon, xenon or krypton; a radioactive gas such as Xe¹³³ or Kr⁸¹; a hyperpolarized noble gas, a low molecular weight hydrocarbon such as methane, ethane, propane, butane, isobutane, pentane or isopentane; a cycloalkane such as cyclobutane or cyclopentane; an alkene such as propene, butene or isobutene; or an alkyne such as acetylene; an ether; a ketone; an ester; halogenated gases, preferably fluorinated or perfluorinated gases, such as fluorinated hydrocarbons; sulphur hexafluoride; perfluoroacetone; perfluorodiethyl ether; perfluoroalkanes; perfluoroalkenes; perfluoroalkynes; perfluorocycloalkanes; and saturated perfluorocarbons. Preferably, the gas is sulphur-hexa fluoride.

In use, diffusion of the gas from the compartment out to the target site causes cavitation of the nanolipobubble (NLB) compromising its structural integrity and, in turn, facilitating release of the drug compound from within the compartment to the target site.

The liposomal shell typically further comprises a polymeric coating at least partially covering the shell, but generally covering the shell in toto. The polymeric coating is usually pH responsive so as to undergo a conformational change and compromise the structural integrity of the coating at pH values lower than physiological pH, more preferably at pH values similar to that of a cancerous tumour, typically about pH 6. Cancerous tumours are known to have a pH lower than that of normal healthy tissue. The polymeric coating may be at least one polymeric coating selected from the group consisting of, but not limited to: biocompatible polymers, ionic polymers preferably anionic and/or cationic polymers. The ionic polymers may include but are not limited to: gelatin, polyethyleneimine (PEI), poly-L-lysine (PLL), carrageenan, pectin, sodium alginate, carboxylic polymers, sulfate, and amine functionalized polymers such as polyacrylic acid (PAA), polymethacrylic acid, polyethylene amine, polysaccharides such as alginic acid, pectinic acid, carboxy methyl cellulose, hyaluronic acid, heparin (mucopolysaccharide), chitosan, carboxymethyl chitosan, carboxymethyl starch, carboxymethyl dextran, heparin sulfate, chondroitin sulfate, cationic guar, cationic starch, and their salts, poly (butyl cyanoacrylate) (PBCA), poly(lactic acid) (PLA), poly(propylene fumarate)(PPF), polyanhydrides.

In a preferred embodiment of the invention the polymeric coating is a cationic polymer, further preferably chitosan.

In another embodiment of the invention the liposomal shell is coated with two or more coatings being sequentially layered. The two or more coatings which are sequentially layered preferably alternate between a cationic polymer coating and an anionic polymer coating. The cationic polymer coating is preferably chitosan, and the anionic polymer coating is preferably polyacrylic acid.

The liposomal shell may further comprise a lyoprotectant. Preferably, the lyoprotectant may be a sugar. The sugar may be at least one sugar selected from the group consisting of, but not limited to: lactose and fructose.

In a preferred embodiment of the first aspect of the invention there is provided for a nanoliposomal drug delivery system comprising: a nanoliposomal shell consisting of distearoyl phosphocholine (DSPC), distearoyl phosphatidylethanolamine-m-polyethylene glycol (DSPE-m-PEG) and a surfactant, the shell defining an inner compartment; and a drug compound housed inside the inner compartment defined by the nanoliposomal shell. The nanoliposomal shell typically further comprises a gas housed within the inner compartment so as to form a nanolipobubble (NLB). The nanoliposomal shell and/or the nanolipobubble typically further comprises a polymeric coating at least partially covering the shell, but generally covering the shell in toto.

According to a second aspect of this invention there is provided a liposomal drug delivery system (LDDS) for the release of at least one drug compound at a target site in a human or animal body, the liposomal drug delivery system comprising a liposomal shell consisting of distearoyl phosphocholine (DSPC) and cholesterol (CHO), the shell defining an inner compartment. The distearoyl phosphocholine (DSPC) is typically 1,2-distearoyl-sn-glycero-3-phosphocholine.

The liposomal drug delivery system typically further comprises a drug compound housed inside the inner compartment defined by the liposomal shell. The drug compound may be at least one drug compound selected from the group consisting of, but not limited to amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, antiparkinson agents, antidepressants, antineoplastic agents, immunosuppressants, antiviral agents, antibiotic agents, antifungal agents, antimicrobial agents, appetite suppressants, antiemetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds, oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol-lowering agents, phytochemicals, and antioxidants. The drug classes mentioned above are listed for illustrative purposes, the liposomal drug delivery system (LDDS) according to the invention may include any pharmaceutical formulation regardless of the active substance and/or substances incorporated therein.

Preferably the drug is at least one anti-neoplastic drug selected from the group consisting of, but not limited to: camptothecin, taxanes and platinum compounds, preferably the anti-neoplastic drug is camptothecin. In embodiments wherein the drug is camptothecin, the compartment provides protection for the housed drug from lactone ring opening typically taking place at physiological conditions in use. The non-polar groups of the liposomal shell facilitates housing non-polar drugs such as camptothecin (CPT), therein preventing drug leakage from the liposomal shell prior to the liposomal shell reaching the target site, in use.

The liposomal shell typically further comprises a surfactant. The surfactant may be is at least one surfactant selected from the group consisting of, but not limited to: dioctyl sulfosuccinate (DOS), Tween 80 and Span 80, or any combination thereof, preferably the surfactant is dioctyl sulfosuccinate (DOS). The surfactant may in use increase the stability of the liposomal shell. The surfactant is typically adsorbed into or onto the liposomal shell. The higher the concentration of the surfactant in the liposomal shell the better the stabilization and the smaller the liposomal shells formed. The surfactant facilitates manufacturing liposomal shells having nano dimensions.

The liposomal shell is generally configured such that non-polar functional groups of the distearoyl phosphocholine (DSPC) and the cholesterol (CHO) are directed inwardly toward the compartment and polar functional groups directed outwardly toward an outer surface of the shell. In use, the non-polar functional groups of the liposomal shell increases the solubilisation of non-polar and/or lipophilic drug compounds such as camptothecin (CPT) housed within the compartment.

The target site is typically cancerous cells located in or on the human or animal body, preferably cancerous cells that are formed into a tumour, further preferably the tumour being an ovarian tumour.

The liposomal shell may have a diameter of less than about 200 nm, preferably less than about 160 nm. The liposomal shell may be sized so as to form a nanoliposome (NLS). In use, nanoliposomes increase the enhanced permeability and retention (EPR) effect, therein facilitating increased drug delivery to the target site. Liposomal shells having a diameter of about 200 nm, preferably less than about 160 nm, will facilitate successful targeting of the liposomal shells to the tumour.

The nanoliposome (NLS) typically further contains a gas housed within the inner compartment defined by the shell so as to form a nanolipobubble (NLB) and thus a nano-lipobubble liposomal drug delivery system (NLB-LDDS). The gas may be at least one gas selected from the group consisting of, but not limited to: air, nitrogen, oxygen, carbon dioxide, hydrogen, nitrous oxide, a noble or inert gas such as helium, argon, xenon or krypton; a radioactive gas such as Xe¹³³ or Kr⁸¹; a hyperpolarized noble gas, a low molecular weight hydrocarbon such as methane, ethane, propane, butane, isobutane, pentane or isopentane; a cycloalkane such as cyclobutane or cyclopentane; an alkene such as propene, butene or isobutene; or an alkyne such as acetylene; an ether; a ketone; an ester; halogenated gases, preferably fluorinated or perfluorinated gases, such as fluorinated hydrocarbons; sulphur hexafluoride; perfluoroacetone; perfluorodiethyl ether; perfluoroalkanes; perfluoroalkenes; perfluoroalkynes; perfluorocycloalkanes; and saturated perfluorocarbons. Preferably, the gas is sulphur-hexa fluoride.

In use, diffusion of the gas from the compartment out to the target site causes cavitation of the nanolipobubble (NLB) compromising its structural integrity and, in turn, facilitating release of the drug compound from within the compartment to the target site.

The liposomal shell typically further comprise a polymeric coating at least partially covering the shell. The polymeric coating may be pH responsive so as to undergo a conformational change and compromise the structural integrity of the coating at pH values lower than physiological pH, more preferably at pH values similar to that of a cancerous tumour, typically about pH 6. The polymeric coating may be at least one polymeric coating selected from the group consisting of, but not limited to: biocompatible polymers, ionic polymers preferably anionic and/or cationic polymers. The ionic polymers may include but are not limited to: gelatin, polyethyleneimine (PEI), poly-L-lysine (PLL), carrageenan, pectin, sodium alginate, carboxylic, sulfate, and amine functionalized polymers such as polyacrylic acid (PAA), polymethacrylic acid, polyethylene amine, polysaccharides such as alginic acid, pectinic acid, carboxy methyl cellulose, hyaluronic acid, heparin (mucopolysaccharide), chitosan, carboxymethyl chitosan, carboxymethyl starch, carboxymethyl dextran, heparin sulfate, chondroitin sulfate, cationic guar, cationic starch, and their salts, poly (butyl cyanoacrylate) (PBCA), poly(lactic acid) (PLA), poly(propylene fumarate)(PPF), polyanhydrides.

In a preferred embodiment of the invention the polymeric coating is a cationic polymer, further preferably chitosan.

In another embodiment of the invention the liposomal shell is coated with two or more coatings being sequentially layered. The two or more coatings which are sequentially layered preferably alternate between a cationic polymer coating and an anionic polymer coating. The cationic polymer coating is preferably chitosan (CHT), and the anionic polymer coating is preferably polyacrylic acid (PAA).

The liposomal shell may further comprise a lyoprotectant. Preferably, the lyoprotectant may be a sugar. The sugar may be at least one sugar selected from the group consisting of, but not limited to: lactose and fructose.

In a preferred embodiment of the second aspect of the invention there is provided for a nanoliposomal drug delivery system comprising: a liposomal shell consisting of distearoyl phosphocholine (DSPC), cholesterol (CHO), and a surfactant, the shell defining an inner compartment; and a drug compound housed inside the inner compartment defined by the liposomal shell. The nanoliposomal shell typically further comprises a gas housed within the inner compartment so as to form a nanolipobubble (NLB). The nanoliposomal shell and/or the nanolipobubble typically further comprises a polymeric coating at least partially covering the shell, but generally covering the shell in toto.

According to a third aspect of this invention there is provided for use of a liposomal shell for the delivery of a drug compound to a target site in a human or animal body in the treatment and/or diagnosis of a disease, the liposomal shell consisting of distearoyl phosphocholine (DSPC) and distearoyl phosphatidylethanolamine-m-polyethylene glycol (DSPE-m-PEG), the shell defining an inner compartment. The liposomal shell typically further comprises a drug compound housed inside the inner compartment, typically camptothecin (CPT). The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a fourth aspect of this invention there is provided for use of a liposomal shell in the manufacture of a medicament for the delivery of a drug compound to a target site in a human or animal body in the treatment of a disease, the liposomal shell consisting of distearoyl phosphocholine (DSPC) and distearoyl phosphatidylethanolamine-m-polyethylene glycol (DSPE-m-PEG), the shell defining an inner compartment. The medicament is generally formulated as an intravenous (IV) formulation. The liposomal shell typically further comprises a drug compound housed inside the inner compartment. The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a fifth aspect of this invention there is provided for use of a liposomal shell for the delivery of a drug compound to a target site in a human or animal body in the treatment and/or diagnosis of a disease, the liposomal shell consisting of distearoyl phosphocholine (DSPC) and cholesterol (CHO). The liposomal shell typically further comprises a drug compound housed inside the inner compartment, typically camptothecin (CPT). The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a sixth aspect of this invention there is provided for use of a liposomal shell in the manufacture of a medicament for the delivery of a drug compound to a target site in a human or animal body in the treatment of a disease, the liposomal shell consisting of distearoyl phosphocholine (DSPC) and cholesterol (CHO). The medicament is generally formulated as an intravenous (IV) formulation. The liposomal shell typically further comprises a drug compound housed inside the inner compartment. The disease may be cancer, and may be at least one cancer consisting of the group, but not limited to: breast cancer, gastric cancer, colorectal cancer, colon cancer, cancer of the pancreas, non small cell lung cancer, small cell lung cancer, brain cancer, liver cancer, renal cancer, prostate cancer, bladder cancer, ovarian cancer, and hematological malignancies such as leukemia, lymphoma, and multiple myeloma. Preferably, the cancer is ovarian cancer.

According to a seventh aspect of this invention there is provided for a method of treating cancer, preferably ovarian cancer, by administering to a human or animal in need of cancer treatment a liposomal drug delivery system (LDDS) in accordance with a first and/or second aspect of this invention.

According to an eighth aspect of this invention there is provided a method of manufacturing the liposomal drug delivery system (LDDS) according to the first aspect of this invention, the method comprising the steps of:
adding DSPC and DSPE-m-PEG to an organic solvent, preferably a mixture of chloroform and methanol, to produce Solution 1;
adding a surfactant, preferably DOS, to Solution 1 to form Solution 2;
adding a drug compound, preferably CPT, to Solution 2 to form Solution 3;
adding phosphate buffered saline (PBS) to Solution 3 to form Solution 4; and
evaporating Solution 4 under vacuum to produce an aqueous solution of the LLDS.

According to a ninth aspect of this invention there is provided a method of manufacturing the liposomal drug delivery system (LDDS) according to the second aspect of this invention, the method comprising the steps of:
adding DSPC and cholesterol (CHO) to an organic solvent, preferably a mixture of chloroform and methanol, to produce Solution 1;
adding a surfactant, preferably DOS, to Solution 1 to form Solution 2;
adding a drug compound, preferably CPT, to Solution 2 to form Solution 3;
adding phosphate buffered saline (PBS) to Solution 3 to form Solution 4; and
evaporating Solution 4 under vacuum to produce an aqueous solution of the LLDS.

In a preferred embodiment of this invention there is provided for a nano-lipobubble liposomal drug delivery system (NLB-LDDS), comprising bio-responsive and/or biocompatible and/or biodegradable polymers, phospholipids and a gas for the targeted treatment of ovarian cancer following intravenous administration. Anti-neoplastic drug model, camptothecin (CPT), and possibly adjuvant therapeutics and/or phytochemicals will be incorporated in the NLB-LDDS and will be released at the tumour site as a result of passive targeting subsequent to intravenous administration. One such phytochemical is silibinin (SB) a naturally occurring polyphenol antioxidant extracted from the crude seed extract of the milk thistle plant.

The nano-scale dimensions of the NLB-LDDS according to both the first and second aspects of the invention, their specific chemico-physical characteristics imparted due to their unique chemical composition, in conjunction with the micro-physiological phenomenon displayed by tumour tissue, termed the Enhanced Permeability and Retention (EPR) effect, is responsible for the accumulation of the anti-neoplastic nano-lipobubbles at the tumour site, thereby leading to a concentrated release of drug at and accumulation within the tumour tissue enhancing anti-neoplastic efficacy. These NLB-LDDSs also significantly reduce the usual side-effects of CPT seen in existing dosage forms. The drug compound(s) (CPT and SB) are released by the diffusion of the gaseous core which will result in cavitation of the NLB and eventual release of the CPT at the tumor. Furthermore, the effect of the micro-environmental physiological conditions of tumour tissue (e.g. lower pH relative healthy tissue) on the bio-responsive polymer coating of the NLBs enhances drug release following accumulation within tumour tissue.

Release of the drug in the systemic circulation is retarded prior to reaching the tumour site since the drug is encapsulated within the NLB-LDDS, hindering the unfavourable generally widespread biodistribution responsible for the devastating side-effects associated with anti-neoplastic therapy. The NLB-LDDS allows for a concentrated release of CPT and SB at a cancerous tumour site within the human or animal body. The NLB-LDDS drastically improves the therapeutic outcome of ovarian cancer therapy, shortens duration of therapy, improve the health-related quality of life of the patient during therapy and increases the overall five-year survival rate. Furthermore, the targeted drug release facilitated by the NLB-LDDS reduces the overall quantity of drug required to achieve maximal efficacy, as well as hospitalisation and treatment required for the associated side-effects, ultimately reducing the total high costs related to cancer chemotherapy.

Housing lipophyllic drug compounds (for example CPT and SB) inside its compartment the NLB-DDS increases solubility of CPT and SB, and the nano-dimensions (typically caused by the surfactant) increases the EPR effect ensuring the NLB-DDS reaches the target site where the CPT and/or SB can readily contact the tumour. The nano-scale size range of the NLB-LDDS allows the NLB-DDS to circumvent the reticulo-endothelial system, reducing its clearance from the body. The high surface-area:volume ratio afforded by the size and architecture of the NLB-LDDS and the lipid component of the NLBs improves solubilisation of CPT and SB and enhances absorption and bioavailability of CPT and SB (and potentially other anti-neoplastic drugs)

The combined effect of enhanced EPR effect, enhanced solubilisation, enhanced absorption, enhanced bioavailability and decreased clearance from the body all increase the concentration of drug within tumour tissue and, consequently, improves the anti-tumour efficacy of the anti-neoplastic drug.

CPT had shown promise in cancer treatment owing to its anti-neoplastic activity, however, its use was complicated by poor solubility and bad side effects. The NLB-LDDS improves the solubility of CPT which normally displays very poor solubility in aqueous as well as in most organic solvents, which poses an initial challenge in regard to pharmaceutical formulation and administration of CPT (Hatefi and Amsden, 2002; Lui et al., 2009). CPT exhibits a deleterious side-effect profile, which has severely diminished its clinical usefulness (Fan et al., 2010). Structure-activity relationship (SAR) studies have highlighted an active lactone group which is responsible for the insolubility and physiologically-labile properties of CPT, yet crucial to its anti-neoplastic activity (Hatefi and Amsden, 2002; Fan et al., 2010). At physiological pH and above, ring-opening of this lactone group occurs, resulting in reversible conversion to an inactive carboxylate form (Hatefi and Amsden. 2002). This compromises the bioavailability of the active lactone form. Moreover, human serum albumin (HSA) has a particular affinity for the carboxylate form of CPT. Binding to HSA unfavourably affects the lactone-carboxylate equilibrium, further compromising the bioavailability of the active lactone form of CPT (Lui et al., 2009). The housing of CPT inside the compartment of the NLB-DDS helps overcome the severe side effects generally associated with CPT.

As explained above, the NLB-LDDS according to the invention will have a substantially favourable impact on the solubilisation of CPT and also SB, whilst enabling the maintenance of the IV route of administration. Furthermore, the NLB-LDDS will function to protect CPT from the aqueous environment and, as such, from conversion to the inactive carboxylate form. The passive targeting functionality of the NLB-LDDS will favourably alter the biodistribution of CPT, thereby drastically reducing the side-effects that have compromised the clinical usefulness of this potent anti-neoplastic drug. The NLB-LDDSs according to the invention aim to re-establish the use of CPT in the treatment of cancer, particularly ovarian cancer, by capitalising on the advantages of nanotechnology to improve the efficacy and reduce the side-effects of CPT.

The invention is further described, illustrated and/or exemplified below by non-limited embodiments of the invention.

1. Nanoliposomes (NLS) in Accordance with the First and Second Aspects of the Invention

### 1.1 Materials

Camptothecin (CPT) (≧90% purity; Mw=348.35), the model anti-neoplastic drug, the phospholipids employed were 1,2-distearoyl-sn-glycero-3-phosphocholine (DSPC) (≧99% purity; Mw=790.15) and L-α-distearoylphosphatidylethanolamine-methoxy-polyethylene glycol conjugate (DSPE-m-PEG) (≧98%; Mw==2748.1) as well as cholesterol (CHO) (≧99% purity, Mw=386.65) were procured from Sigma Chemical Company (St Louis, Mo., USA). Dioctyl sulfosuccinate sodium salt (DOS) (≧99% purity; MW=444.56) was used as a surfactant and sulphur-hexafluoride (SF₆) was incorporated as the gaseous phase of the liposomal drug delivery system (LDDS). The aforementioned were purchased form Sigma Chemical Company. Chloroform, methanol, buffer salts and all other reagents were of analytical grade and used without further modification. In addition, all A-grade glassware and double de-ionized water was employed in the preparation of formulations.

### 1.2 Methods

Nano-liposomes (NLS) were initially formulated to generate a design of feasible formulations by a Two-Factor, Three-Level, Face-Centered Central Composite Design mathematical model. The nano-liposomes of this design were characterized for optimisation.

### 1.2.1 Formulation of Camptothecin-Loaded Nano-Liposomes (NLS)

Nano-liposomal formulations were formulated by an adapted reverse-phase solvent evaporation method in order to manufacture the liposomal drug delivery systems (LDDSs) according to the first and second aspect of this invention.

Briefly, DSPC (10-30 mg) and either (1) DSPE-m-PEG (10-30 mg) or (2) CHO (10-30 mg), to a total of 40 mg, were dissolved in chloroform:methanol (9:1; 10 mL) under agitation by means of a magnetic stirrer at 400 rpm for 5 minutes, resulting in solutions with weight ratios ranging from 1:3-3:1 of DSPC:DSPE-m-PEG or DSPC:CHO. DOS and CPT were subsequently dissolved in the organic solution. Phosphate buffered solution (PBS) (pH 7.4, 25° C.; 10 mL) was subsequently added to the organic solution under ultra-sonication (Amplitude=80%; 90 seconds), over an ice-bath, employing a Vibracell probe ultrasonicator (Sonics & Materials Inc, Newtown, Conn., USA). This culminated in a formation of a homogenous, single-phase emulsion. Subsequently this emulsion was subjected to evaporation under vacuum (65-75° C.) in a round-bottom flask for 2-4 hours, employing a Multivapor™ (Buchi Labortechnik AG, Switzerland). PBS (pH 7.4, 25° C.; 10 mL) was added periodically during the evaporation process and the formulation was subjected to ultra-sonication as previously outlined for 30 seconds, after each addition. Complete evaporation of the solvent resulted in an aqueous suspension of nano-liposomal drug delivery systems in accordance with either a first or second aspect of this invention. Therefore, nano-liposomes (NLS) according to the first aspect of this invention (DSPE-m-PEG-NLS) and nano-liposomes according to the second aspect of this invention (CHO-NLS) were manufactured.

### 1.2.2 Determination of Nano-Liposomal Size and Zeta Potential

The aqueous nano-liposomal suspension was analyzed for size, and size distribution data employing a Zetasizer NanoZS (Malvern Instruments Ltd, Malvern, Worcestershire, UK). Samples were filtered through a 0.22 μm filter into a suitable cuvette and analyzed by dynamic light scattering, enhanced by a non-invasive back scatter technology, to produce size and size distribution profiles based on the diffusion of particles in the sample by Brownian motion. Measurements were derived from 2 angles, thereby increasing the accuracy of the measurements. All size measurements were conducted in triplicate at 25° C. over a three hour period, whilst being maintained at 37° C. in an orbital shaker bath (20 rpm).

Briefly, the Zetasizer NanoZS system employs a Laser Doppler Micro-electrophoresis technique to determine the velocity of the particles in the sample in response to an applied electric field. This enables the elucidation of electrophoretic mobility and hence zeta potential of the sample. As outlined above, all zeta potential measurements were conducted in triplicate at 25° C. over a three hour period, whilst the sample was maintained at 37° C. in an orbital shaker bath (20 rpm).

### 1.2.3 Elucidation of Drug Incorporation Efficiency (DIE)

The efficiency of drug incorporation into the compartment of the LDDS was determined by a novel method derived for this LDDS. The model drug used (CPT) is very poorly water soluble. The nano-liposomes (NLS) are ultimately suspended in an aqueous phase. Hence it was hypothesized that the nano-liposomes (NLS) would orientate with the non-polar group of the phospholipids directed toward the core of the nano-liposomes and the polar group directed outwards, towards the aqueous suspending medium. CPT will therefore either be incorporated within the nano-liposomes, or will precipitate out. Unincorporated drug, due to insolubility in the suspending medium, will be present primarily as a precipitate. Therefore, it was rationalised that the unincorporated precipitated drug could be removed by double filtration through a 0.22 μm filter.

Following rotary evaporation to produce an aqueous nano-liposomal suspension, the suspension was double filtered through 0.22 μm filters to remove free drug. The filtrate was subsequently sonicated (Amplitude=80%, 10 minutes) and, thereafter, dissolved in DMSO (1:1). Drug incorporation efficiency was elucidated, in triplicate, using UV-spectroscopy at 366% (Cecil CE 3021, Cecil Instruments Ltd., Milton, Cambridge, UK), with reference to constructed standard curves. The following equation was used to calculate the drug incorporation efficiency as a percentage of the total drug initially added during formulation:

$$Drug Incorporation Efficiency ( DIE ) ( \% ) = \frac{Quantity of incorporated drug}{Quantity of total drug added} \times 100 \qquad Equation 1$$

### 1.2.4 In Vitro Drug Release Analysis

Following removal of free drug from the nano-liposomal suspension, 10 mL samples were enclosed in treated dialysis tubing (cutoff=12000 kDA) and suspended in PBS (pH 7.4, 25° C.; 200 mL). The receptacle was maintained at 37° C. in an orbital shaker bath at 20 rpm. At pre-determined intervals, 5 mL aliquots were removed from the external PBS phase and added to DMSO (5 mL), creating a 1:1 ratio, to prevent precipitation of the drug. Fresh buffer (5 mL) was replaced to the external phase to maintain sink conditions. Vortexed samples were analyzed by UV-spectroscopy at 366, against previously constructed standard curves of CPT in PBS:DMSO (1:1).

### 1.2.5 Morphological Characterization of Nano-Liposomes

The morphology of the nano-liposomes (NLS) was assessed by two imaging modalities. The shape and size of the nano-liposomes (NLS) were, initially, confirmed by Transmission Electron Microscopy (TEM). Briefly, copper grids were coated with the nano-liposomal suspension, using a micro-pipette and allowed to dry for approximately one hour. The grids were then inserted into the loading chamber of a Transmission Electron Microscope (TEM). TEM employs energized electron beams to produce high resolution images at significantly high magnifications. Photomicrographs were obtained at different magnifications to illustrate the structure of individual nano-liposomes.

In addition to the aforementioned imaging technique, micro-ultrasound imaging employing a Vevo® 2100 (Visualsonics, Toronto, Ontario, Canada) was employed to confirm the overall appearance of the nano-liposomes. This technique, further, highlights behavioural characteristics such as aggregation of the nano-liposomes, which is a vital indicator of stability. A 10% ^{w}/ᵥ carrageenan hydrogel was prepared, onto which ultra-sound gel was applied. The nano-liposomal suspension was injected into the hydrogel and an ultra-sound beam was applied, producing images of the nano-liposomes as they dispersed through the hydrogel.

### 2. Results and Discussion Regarding the Nano-Liposomal Drug Delivery System (NLS-DDS)

### 2.1 Zeta-Size Analysis of Nano-Liposomes

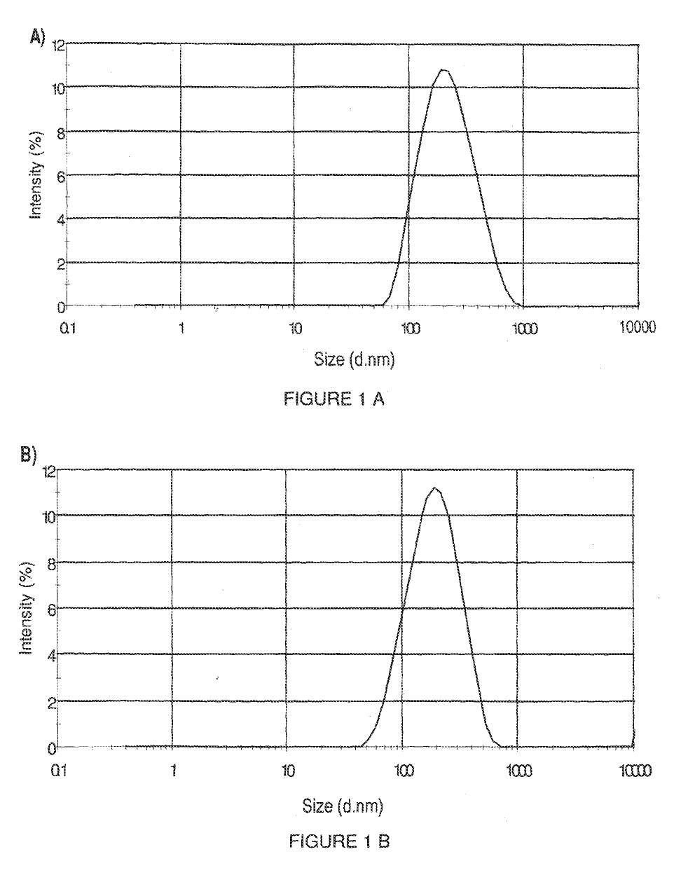

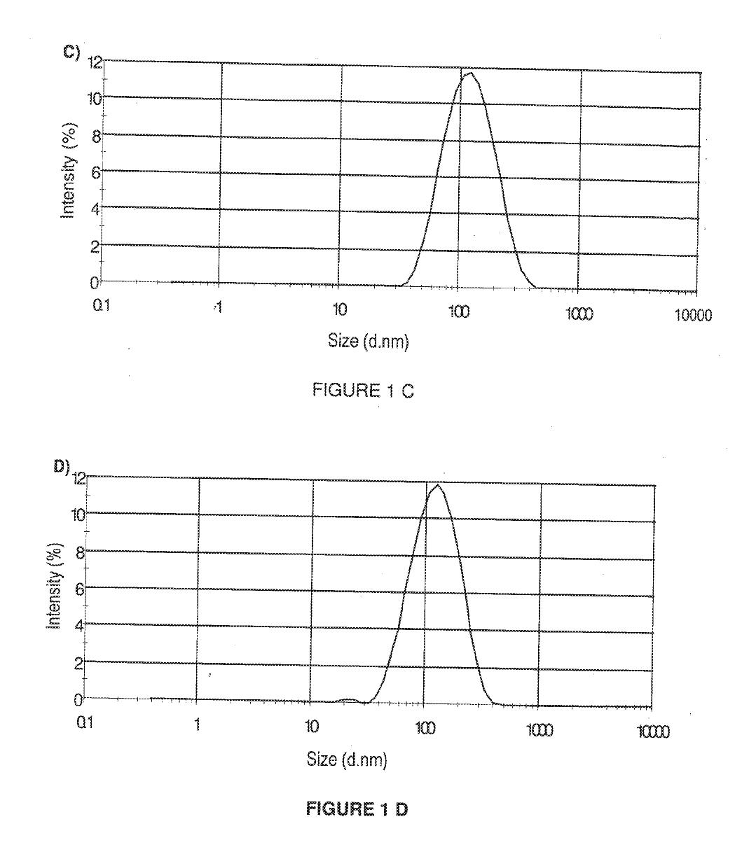

As previously outlined, the benefits of the LDDS presented rely in part on the nano-scale size of the nano-liposomes (NLS). The nano-size scale will be in part responsible for the targeting nature of the LDDS. Furthermore, nano-sizing enables the solubilisation of the poorly aqueous soluble anti-neoplastic drugs. Hence, assessment nano-liposomal size was fundamentally important. A benchmark size of about 200 nm, preferably about 160 nm was established. All formulations fell within this size range. The foremost contributor to size variations between the various formulations was concentration of DOS. Higher concentrations of DOS resulted in a reduction of nano-liposome size as well as a narrower size distribution, indicated by the lower Polydispersity Index (PdI). Generally, the surfactant is adsorbed into or onto the liposomal surface forming a part of the liposomal structure. The higher the concentration of surfactant, the more surfactant available for adsorption into or onto the liposomal surface, the better the stabilization of the overall LDDS and the smaller the overall LDDS. In addition, CHO-containing nano-liposomal formulations were generally of a larger size than DSPE-m-PEG-containing nano-liposomal formulations, as indicated in FIG. 1. This was attributed to the bulkiness on the CHO molecule.

FIG. 1 shows typical Zeta Size profiles (Size vs Intensity) of a DSPC:CHO nano-liposomal drug delivery system at T=0 hours (a) and T=3 hours (b) in accordance with a second aspect of this invention (CHO-NLS). FIG. 1 also shows typical Zeta Size profiles (Size vs Intensity) of a DSPC:DSPE-m-PEG nano-liposomal drug delivery system at T=0 (c) and T=3 (d) in accordance with a first aspect of this invention (DSPE-m-PEG-NLS).

### 2.2 Surface Charge Characterization of Nano-Liposomes

Zeta potential is an indication of the surface charge of the nano-liposomes formulated, and hence the propensity of these nano-liposomes (NLS) for aggregation. Zeta potential is thus considered a suitable indicator of formulation stability. All nano-liposomal formulations displayed negative zeta potentials, which were attributed to the anionic nature of the surfactant (DOS). However, a significant difference was noted between DSPE-m-PEG-containing and CHO-containing formulations (i.e. the first and second aspects of the invention respectively). A nano-liposomal drug delivery system comprising the combination of DSPC and CHO, according to a second aspect of this invention (CHO-NLS), exhibited a significantly more negative zeta potentials compared to a nano-liposomal drug delivery system comprising the combination of DSPC:DSPE-m-PEG, according to a first aspect of this invention (DSPE-m-PEG-NLS) (˜−45 mV and ˜7 mV respectively). Furthermore, the strong surface charge (i.e. high zeta potential) exhibited by the drug delivery system according to a second aspect of the invention increases the potential for coating with cationic polymers.

### 2.3 Evaluation of Drug Incorporation Efficiency (DIE)

Achieving adequately high levels of drug incorporation into nano-liposomal drug delivery systems was particularly challenging due to the nano-scale size range of these LDDSs. DSPC:CHO nano-liposomes (according to a second aspect of this invention), CHO-NLS, demonstrated favourably high DIE, with all, except two, formulations displaying DIE>60%. The maximum reproducible DIE achieved was 81.47%.

TABLE 1

| | | DSPE-m- | | |
|---|---|---|---|---|
| DSPC (% ^{w}/ᵥ) | CHO (% ^{w}/ᵥ) | PEG (% ^{w}/ᵥ) | DOS (% ^{w}/ᵥ) | DIE (%) |
| 0.1 | 0.3 | | 0.1 | 55.50 |
| | | | 0.2 | 62.79 |
| | | | 0.3 | 64.42 |
| | | 0.3 | 0.1 | 28.45 |
| | | | 0.2 | 32.46 |
| | | | 0.3 | 76.31 |
| 0.2 | 0.2 | | 0.1 | 39.21 |
| | | | 0.2 | 62.59 |
| | | | 0.3 | 75.84 |
| | | 0.2 | 0.1 | 37.95 |
| | | | 0.2 | 36.72 |
| | | | 0.3 | 37.02 |
| 0.3 | 0.1 | | 0.1 | 60.94 |
| | | | 0.2 | 64.85 |
| | | | 0.3 | 81.47 |
| | | 0.1 | 0.1 | 28.35 |
| | | | 0.2 | 44.19 |
| | | | 0.3 | 43.16 |

### 2.4 In Vitro Drug Release Analysis

The LDDSs in accordance with first and second aspects of this invention release all incorporated drug in less than 24 hours. Drug release profiles have highlighted sufficient time for accumulation of the LDDS within tumour tissue, before adequate CPT is released. This has a substantial impact on the anti-tumour efficacy of the drug, maintenance of the stability of the lactone ring of CPT, as well as, the detrimental side-effects that have limited clinical use of this drug. Furthermore, once the LDDS has accumulated within the tumour tissue, rapid drug release may prove favourable in over-coming saturable mechanisms of drug resistance. DOS concentration appeared to have the most significant effect on drug release kinetics. It was hypothesized that the stabilizing effect of the surfactant, as evidenced by the direct relationship of Zeta Potential to DOS concentration, retards drug release to some extent, resulting in a larger MDT.

The LDDS comprises a liposomal shell which defines therein a compartment. Incorporation of a gas of low diffusibility into the compartment, such as sulphur hexa-fluoride (SF₆), as well as a polymeric coating further retards CPT release from the LDDS.

Since CPT acts on the S-phase of the cell cycle, prolonged release may prove substantially beneficial to the anti-tumour efficacy of the LDDS. In addition, further limiting the quantity of CPT released prior to accumulation of the LDDS within tumour tissue will result in reduced side-effects and enhanced drug load exerting anti-tumour effects within the tumour tissue.

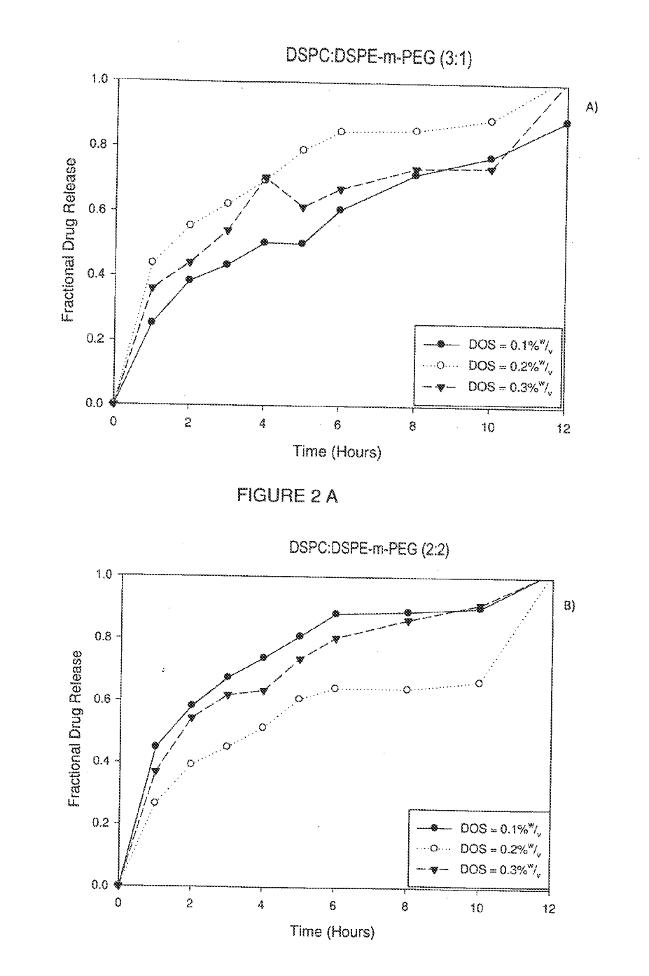

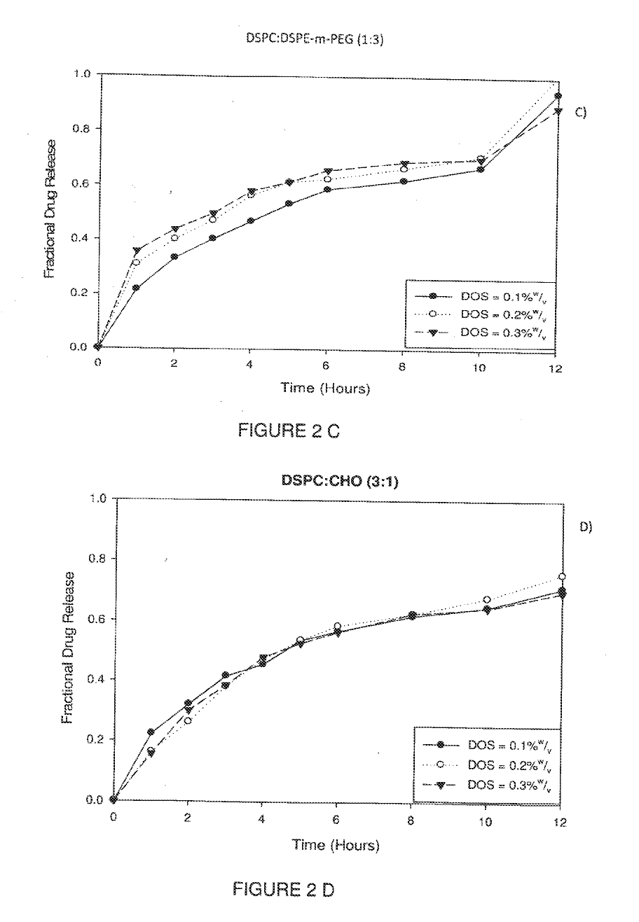

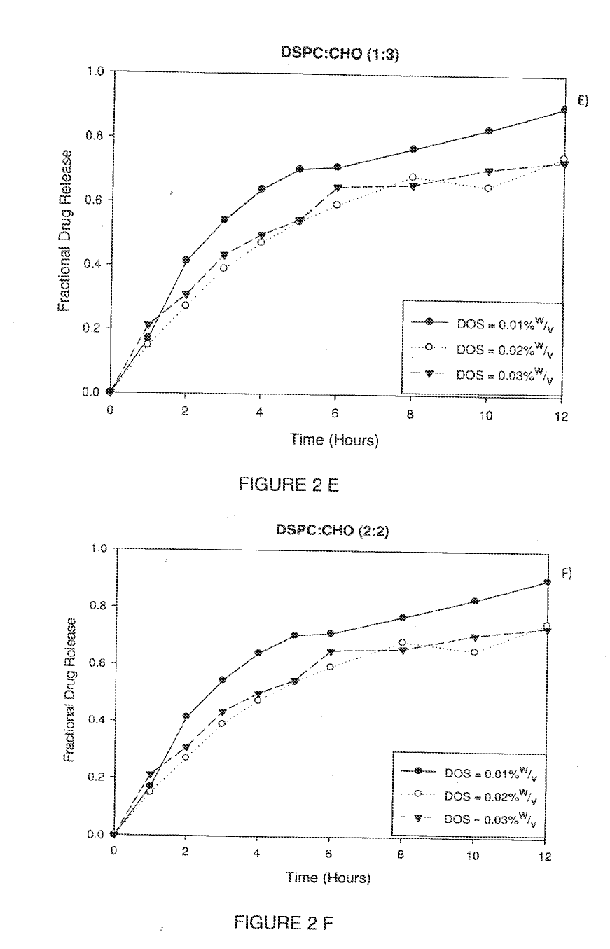

FIG. 2a-c shows fractional drug release for nano-liposomal drug delivery systems (LDDSs) in accordance with a first aspect of this invention with varying DSPC:DSPE-m-PEG ratios (the DSPE is at all times DSPE-m-PEG). FIG. 2d-f shows fractional drug release for nano-liposomal drug delivery systems (LDDSs) in accordance with a second aspect of this invention with varying DSPC:CHO (3:1-1:3) ratios.

### 2.5 Morphological Characterisation of Nano-Liposomes

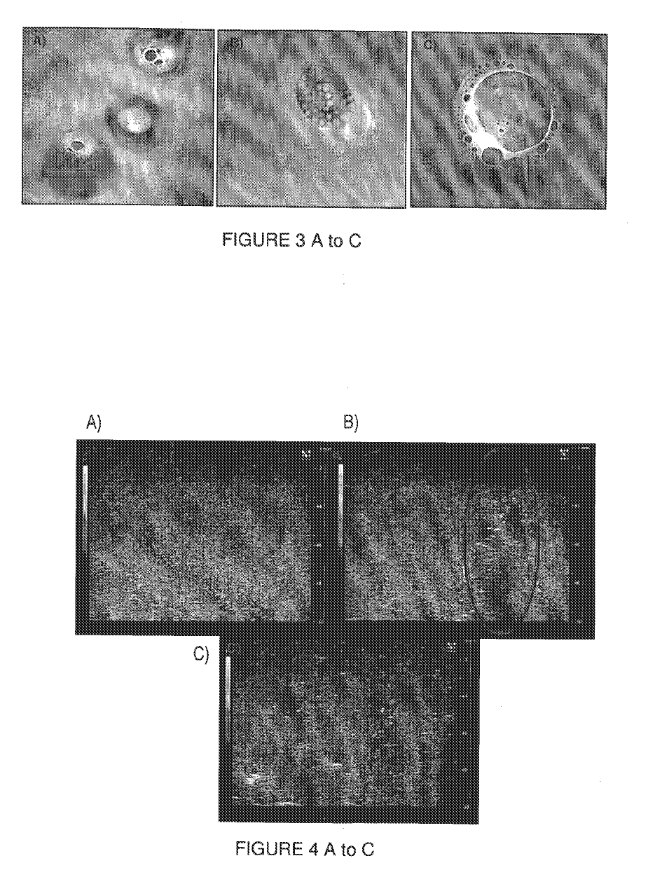

Transmission electron photomicrographs confirmed the presence of regular, well defined, near spherical DSPC: CHO nano-liposomes (CHO-NLS), as indicated in FIG. 3 a-c. FIG. 3 shows transmission electron photomicrographs of nano-liposomes at 30000× magnification (a), 40000× magnification (b) and 50000× magnification (c), respectively.

Micro-ultrasound imaging was especially advantageous in highlighting the dispersion characteristics of formulated nano-liposomes. FIG. 4 denotes the appearance of DSPC: CHO nano-liposomes (in accordance with a second aspect of the invention), CHO-NLS, upon injection into a carrageenan hydrogel and the favourable dispersion of nano-liposomes through the viscous hydrogel medium. The absence of nano-liposomal aggregation is clearly evident, and indicative of appreciable formulation stability. FIG. 4 shows micro-ultrasound images of a) carrageenan hydrogel prior to introduction of nano-liposomes, b) injection of nano-liposomes and v) dispersion of nano-liposomes through the hydrogel 2 minutes post injection.

3. Incorporation of Gas into the NLS to Form a Nano-Lipobubble Liposomal Drug Delivery System (NLB-LDDS)

### 3.1 Preparation of CHO Containing NLS, and DSPE-m-PEG Containing NLS for Gas Incorporation

DSPC, DOS and either CHO or DSPE-m-PEG (concentrations as per Table 2) were simultaneously dissolved in a chlororform:methanol (9:1; 10 mL) solvent system under continuous stirring at 400 rpm for 5 minutes, employing a magnetic stirrer. Camptothecin (CPT) (0.05% ^{w}/ᵥ was added to the organic solution under continuous agitation. Phosphate buffered saline (PBS) (pH 7.4, 25° C.; 10 mL) was subsequently added to the organic solution under ultra-sonication (amplitude=80%; 90 seconds), over an ice-bath, employing a Vibracell probe ultrasonicator (Sonics & Materials Inc., Newtown, Conn., USA). This culminated in the formation of a homogenous, single-phase emulsion. This emulsion was subsequently subjected to evaporation under vacuum (60-65° C.) for 2-3 hours, employing a Multivapor™ (Buchi Labortechnik AG, Switzerland). PBS (pH 7.4, 25° C.; 10 mL) was added periodically during the evaporation process and the formulation was subjected to ultra-sonication as previously outlined for 30 seconds, after each addition. Complete evaporation of the solvent resulted in an aqueous NLS suspension. The resultant NLS suspension was subjected to three cycles of freezing at about 70° C. and thawing at about 37° C., to convert multilamellar NLS to unilamellar NLS with filtration through a 0.22 μm millipore filter after each freeze-thaw cycle. All ensuing modifications and analyses were conducted in triplicate (n=3) on these unilamellar NLS.

TABLE 2
Formulatory composition of the NLS systems obtained by statistical
optimization

| | CHO-NLS | DSPE-m-PEG- |
|---|---|---|
| Materials | (% ^{w}/ᵥ) | NLS (% ^{w}/ᵥ) |
| DSPC | 0.3 | 0.133 |
| DSPE-m-PEG | — | 0.267 |
| CHO | 0.1 | — |
| DOS | 0.3 | 0.3 |

### 3.2 Conversion of Formulated NLS to NLB Via Gas Incorporation: Effect of Sonication Duration

10 mL of the CHO-NLS (according to a second aspect of this invention) and 10 mL of the DSPE-m-PEG-NLS (according to a first aspect of this invention) was filtered and injected into 20 mL vials individually. SF₆ gas was introduced into the headspace of the vials, which vials were subsequently sealed. Sonication of the vials was undertaken in a bath type sonicator causing the SF₆ gas to penetrate the lipid membrane of both the CHO-NLS and DSPE-m-PEG-NLS, and form a gaseous core, thereby creating nano-lipobubbles (NLBs) according to a first aspect of the invention (DSPE-m-PEG-NLB) and nano-lipobubbles (NLBs) according to a second aspect of the invention (CHO-NLB).

Sonication was undertaken for 2, 3 and 5 minutes to determine the effect of sonication duration on the ultimate size and stability of the NLB. The variation in size and zeta potential was insignificant after sonication for 2 and three minutes. However, following sonication for 5 minutes, the PdI was unfavorably higher due to the formation of a small proportion (<5%) of NLB below 25 nm. The zeta potential of the NLB sonicated for 5 minutes exhibited an unfavorable deficit of ˜10 mV for CHO-NLB and ˜4 mV for DSPE-m-PEG-NLB. Hence, sonication duration of 3 minutes was delineated for all further formulations.

### 3.3 Investigating the Effect of Lyophilization on NLB Size and Stability

To determine the effect of lyophilization on the stability of formulated NLB, the average size, size distribution and zeta potential of formulated NLB prepared pre- and post-lyophilization with and without a lyoprotectant was determined CHO-NLS and DSPE-m-PEG-NLS were formulated and converted to NLB as outlined above. The formulated NLB were subjected to size, size distribution and zeta potential analysis in triplicate over a 3 hour period whilst being maintained at 37° C. in an orbital shaker bath rotating at 25 rpm.

Simultaneously, unmodified NLS suspensions (15 mL), as well as NLS suspensions containing lactose or fructose (˜0.05%^{w}/ᵥ) as lyoprotectants were frozen at −70° C. for 48 hours. The samples were subsequently lyophilized (Labconco, Kansas City, Mo., USA) and the products were re-suspended in PBS (pH 7.4; 25° C.; 10 mL) to a concentration of 0.5% ^{w}/ᵥ. The resultant NLS suspensions were subjected to three freeze-thaw cycles with filtration through 0.22 μm millipore filters undertaken after each cycle. Conversion of NLS to NLB was undertaken according to the methodology outlined above. Average size, size distribution and zeta potential analysis ensued over a 3 hour period, whilst the NLB suspensions were maintained at 37° C. in an orbital shaker bath rotating at 25 rpm.

### 3.4 Assessment of Lyoprotectant Efficacy Through Water Content Determination

Determination of water content was undertaken by volumetric Karl Fischer (KF) titration on the lyophilized powder (10 mg) of plain, fructose-containing and lactose-containing formulations employing a Karl Fischer titrator (Mettler Toledo, Columbus, Ohio, USA).

### 3.5 Evaluating Polymeric Coating by Layer-by-Layer (LBL) Self-Deposition

Layer-by-layer (LBL) polymeric coating is based on the principle of electrostatic attraction between oppositely charged molecules, resulting in the alternate deposition of polymers onto charged surfaces. Candidate NLS exhibited an overall anionic surface charge, with CHO-NLS possessing a more strongly negative zeta potential relative DSPE-m-PEG-NLS, which facilitated the establishment of a polycationic primary polymeric layer, followed by the alternate deposition of polyanionic and polycationic polymeric layers.

TABLE 3
Cationic and anionic polymers investigated for application in NLS
coating by the LBL self deposition methodology.

| Cationic Polymers | Concentration (% ^{w}/ᵥ) |
|---|---|
| Gelatin | 0.5-2 |
| PEI | 1-3 |
| PLL | 0.5-3 |
| CHT | 0.1-1.0 |
| Anionic Polymers: | Concentration (% ^{w}/ᵥ)* |
| Carrageenan | 0.2-0.4 |
| Pectin | 0.5-1 |
| Sodium alginate | 0.5-2 |
| PAA | 0.5-2.0 |

Summarily, unilamellar NLS suspension was added drop-wise to a cationic polymer solution under constant agitation employing a magnetic stirrer. Coating was allowed for periods of 3-12 hours under ambient conditions, with zeta potential analysis undertaken at regular intervals to determine successful polymer coating. The cationic NLS suspension was subsequently added drop-wise to an anionic polymeric solution under constant stirring and adsorption of the polymer was allowed under ambient conditions for periods of 6-18 hours, with periodic zeta potential analysis. Two or four polymeric layers were applied. Lactose, a lyoprotectant, was added to the polymer coated-NLS suspension and the suspension was frozen at −70° C. for 48 hours, followed by lyophilization. The lyophilized powder was re-suspended in PBS (pH 7.4; 25° C.) to form polymer coated NLS, and converted to polymer coated NLB as outlined above. Table 3 summarizes the polymers and concentrations thereof investigated as suitable NLS coating materials.

### 3.6 Elucidating the Size Characteristics of Formulated NLS and NLB

The nano-scale size range is central to the clinical relevance and feasibility of the LDDS of this invention. Variation in average size and size distribution were also highlighted as key indicators of formulation stability. Hence, all modifications investigated were initially assessed from the standpoint of the effect the modification had on the resultant size profile of the formulation. During initial investigations to assess the effect of each modification on the size profile on the formulation, analysis was undertaken over a 3 hour period whilst the NLB was maintained at physiological temperature in an orbital shaker bath rotating at 25 rpm. Table 4 succinctly summarizes the modifications investigated for their effect on the average size and size distribution characteristics of the NLS and NLB.

TABLE 4
Summary of average size and size distribution assessments undertaken
on candidate NLS and NLB formulations and the modifications that
ensued
Modifications Assessed by Average Size and Size
Distribution Analyses

Candidate NLS from experimental design
Impact of gas incorporation to create NLB
Effect of sonication time
Influence of lyophilization (with and without
lyoprotectants)
Effect of phytochemical incorporation
Bearing of polymeric coating

### 3.7 Surface Charge Characterization of Formulated NLS and NLB

Size characteristics and surface charge characteristics are of equal importance regarding this invention due to the intravenous (IV) nature of the formulated LDDS and the severe implications of NLB aggregation in vivo. In addition, the high cost of antineoplastic drugs warrant the need for a stable formulation with lengthened shelf-life. Consequently zeta potential determination was undertaken in conjunction with size analysis as described in. In addition variation in zeta potential was a distinct indicator of successful polymeric coating with oppositely charged polymers.

### 3.8 Morphological Characterization of Formulated NLB

Scanning electron microscopy (SEM) was undertaken on the lyophilized products (CHO-NLB and DSPE-m-PEG-NLB) following polymeric coating, employing a Phenom™ scanning electron microscope (FEI Company, Hillsboro, Oreg., USA) to qualitatively assess the resulting morphological structures of lyophilized products. Samples were fixed as a monolayer to a sampling stub and coated with gold-palladium for 30 seconds before photomicrographs were acquired.

Furthermore, lyophilized powders of formulated NLS were reconstituted with phosphate buffered saline (PBS) (pH 7.4; 25° C.) in the presence of fluorescein isothiocyanate (FITC) dye and subsequently converted to NLB, as outlined above. The NLB suspension was allowed to dry on a slide for 1 hour, followed by imaging employing an inverted immunofluorescence microscope (Olympus IX71, Olympus, Tokyo, Japan) after 100 mS exposure.

### 3.9 Investigating the Efficiency of CPT and SB Incorporation

Determination of the efficiency of CPT incorporation was undertaken on candidate NLS for comparison to predicted values, on formulated NLB, following silibinin (SB) incorporation and the application of polymer coating to NLB. Drug incorporation efficiency (DIE) of CPT was undertaken as earlier above.

### 3.10 Generation of a Standard Curve for the Photospectroscopic Quantification of CPT

In addition to physiological pH, drug release was also undertaken at approximate tumoural pH (6.0; 37° C.) to determine the effect of lower pH on CPT release characteristics. The analysis of CPT release characteristics at approximate tumoural pH (6.0) necessitated the construction of a standard curve of CPT in PBS (pH 6.0; 37° C.) to enable photospectroscopic quantification of CPT. Preparation of a stock solution of CPT in DMSO and subsequent serial dilutions were undertaken. Following a wave scan to delineate the optimal wavelength for CPT determination at pH 6.0, the aforementioned serial dilutions were analyzed at 345 nm.

### 3.11 Elucidation of CPT and SB Release Characteristics

Drug release investigations were undertaken at approximate tumoural and physiological pH, following reconstitution of lyophilized powder and conversion to NLB as explicated earlier. Quantification of drug release was undertaken with reference to the relevant standard curves for CPT and SB. Adjustment of the concentration and volume of NLB suspension was undertaken in order to accommodate for the inclusion of SB and maintain sink conditions for both compounds.

### 3.12 Evaluating Formulation Stability

The clinical feasibility and usefulness of formulations is influenced in large part by the stability of formulations under various conditions. Whilst surface charge was denoted as the initial indicator of formulation stability, other conditions that have the potential to affect or be affected by stability of the formulation required further consideration. Hence, stability of formulations was determined through exposure to serum, behavioral changes following reconstitution and long term storage stability.

### 3.13 Stability in the Presence of Serum

For intravenously administered formulations, establishment of the characteristics of the formulation in the presence of serum is a vital determination. Coated and uncoated CHO-NLB and DSPE-m-PEG-NLB (10 mL) were incubated at 37° C. for 1 hour with FBS (50% ^{v}/ᵥ), which is regarded an appropriate concentration to adequately mimic physiological conditions. At 15 minute intervals 100 μL of the NLB-FBS combination was diluted with 10 mL PBS (pH 7.4, 37° C.) and average size, size distribution and surface charge characterization ensued, employing a Zetasizer NanoZS (Malvern Instruments Ltd, Malvern, Worcestershire, UK).

3.14 Assessing Stability of the Formulation after Reconstitution

Assessing the stability of NLB suspensions post-reconstitution is critical to delineate pre-administration storage conditions and the provision period that can be allowed between reconstitution of the formulation and administration to the patient. A Turbiscan™ LAB (Formulaction, L′Union, France) was employed to qualitatively analyze the behavioral characteristics of formulated NLB suspensions. The relevant NLB suspensions (20 mL) were introduced into specialized vials and analyzed at pre-determined intervals over a 12 hour period at 25° C.

### 3.15 Determining the Effect of Long-Term Storage on Physicochemical Characteristics of NLB

The long-term stability of formulated NLB was determined as a function of change in average size, zeta potential, CPT content and SB content over the analysis period of 3 months. Lyophilized NLS were sealed in transparent vials with SF₆ gas filled into the headspace and stored at 4° C. and 25° C. At weekly intervals PBS was introduced into the vials and sonication in a bath-type sonicator was undertaken to form NLB, as outlined above. Drug content, zeta sizing and zeta potential determinations were undertaken.

### 4. Results and Discussion (CHO-NLB-LDDS and DSPE-m-PEG-NLB-LDDS)

### 4.1 Size and Surface Charge Characterization of Candidate Formulations

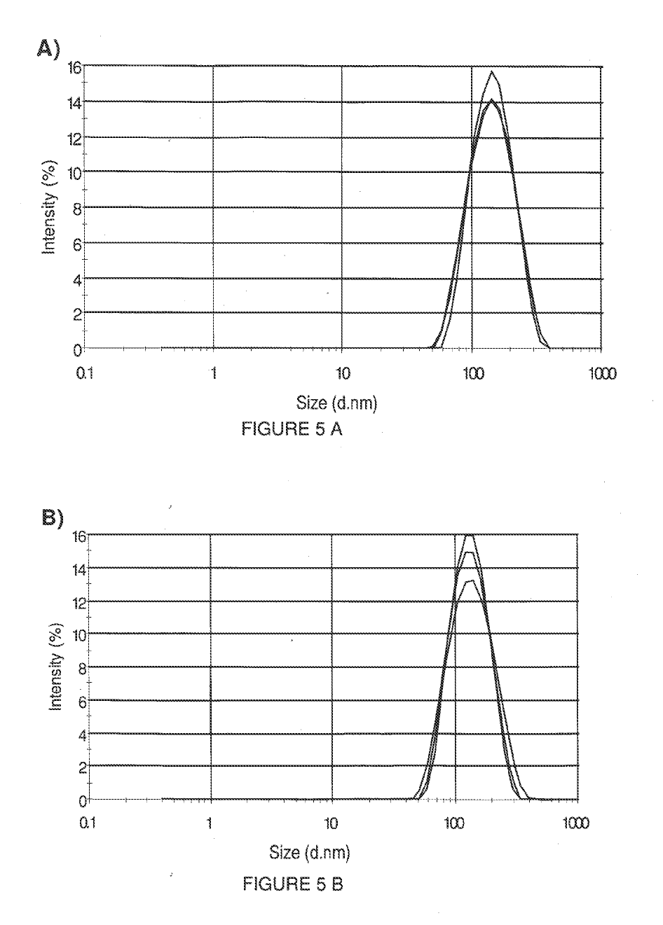

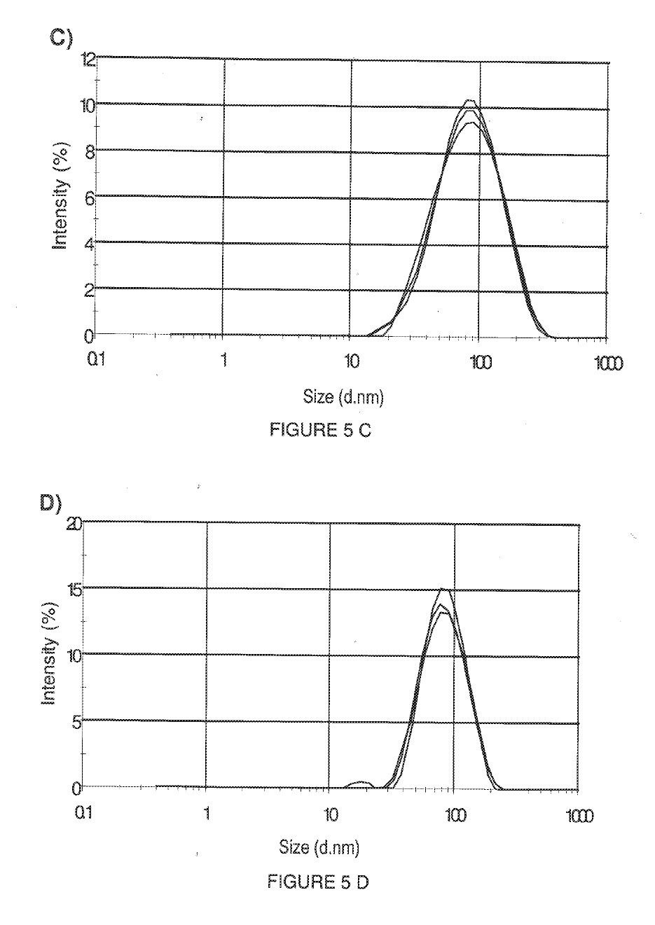

The average size of CHO-NLS was 2.41% larger than predicted by statistical optimization which, given the nano-scale of the formulation, is quite satisfactory. In addition, the average size obtained was still adequately below the benchmark size of about 200 nm, preferably about 160 nm, that was initially delineated for favorable passive targeting to tumour tissue, as indicated in FIG. 5a. The PDI (result not shown) was 0.151, indicating the narrow size distribution of NLS within the formulation. Conversion of NLS to NLB resulted in a slight decrease in average size, illustrated in the size profile in FIG. 5b. This may be attributed to replacement of the aqueous core with a gaseous core which occupied a smaller volume. In addition, ultrasonication employed in creating the gaseous core may have caused a reduction in the average size of the CHO-NLB.

The zeta potential obtained experimentally for the candidate CHO-NLS formulation was 9.26% less negative than that predicted for this formulation by statistical optimization. There was a further unfavorable decrease in surface charge following conversion of the CHO-NLS to CHO-NLB. This may be attributed to slight destabilization of the lipid membrane during the conversion process. However, the zeta potential of formulated CHO-NLB remained highly favorable, designating a stable formulation that is not inclined to aggregation.

The average size and zeta potential of candidate NLS, as well as the average size and zeta potential following conversion of these NLS to NLB is outlined in Table 5. In addition, a comparison to the measured responses predicted by statistical optimization for each of the candidate NLS is highlighted through the percentage deviation value.

TABLE 5
Experimentally determined average size and zeta potential of candidate
CHO-NLS and DSPE-m-PEG-NLS and NLBs, as well as the percentage
deviation from the values predicted for NLS by computational modelling.

| | Average | | Zeta | |
|---|---|---|---|---|
| Formulation | Size (d · nm) | % Deviation | Potential (mV) | % Deviation |
| CHO-NLS | 132.4 | 2.41 | −37.9 | 9.26 |
| CHO-NLB | 125.6 | — | −28.1 | — |
| DSPE-m- | 83.41 | 2.18 | −7.74 | 1.18 |
| PEG-NLS | | | | |
| DSPE-m- | 84.20 | — | −7.44 | — |
| PEG-NLB | | | | |

The average size of candidate DSPE-m-PEG-NLS was 2.18% smaller than predicted for this formulation by statistical optimization, as illustrated in FIG. 5c. Conversion to DSPE-m-PEG-NLB demonstrated an insignificant increase in the average size of the formulation, depicted in FIG. 5d. The exceptional size characteristics of DSPE-m-PEG-NLB greatly favors passive targeting of the LDDS to tumour tissue by the enhanced permeability and retention (EPR) effect and, hence, may tremendously improve the safety and efficacy of CPT delivered by this LDDS in vivo. The zeta potential achieved experimentally for the candidate DSPE-m-PEG-NLS was very closely correlated with the zeta potential predicted for this LDDS by statistical optimization, deviating by only 1.19%. Conversion of the DSPE-m-PEG-NLS to DSPE-m-PEG-NLB resulted in a marginal decrease in surface charge, analogous to that observed for CHO-NLB.

### 4.2 Determination of the Effect of Lyophilization on Formulated NLBs

Long term storage stability has presented a constant challenge, leading to a growing interest in stabilization mechanisms for liposome storage (Chaudhury et al., 2012). This consideration was also relevant herein, whereby the storage form of the LDDSs was envisaged to be NLS in the presence of SF₆, which would form the gaseous core upon conversion to NLB. Hence, lyophilization was investigated as a means of creating a formulation that demonstrates long-term storage viability.

The effect of lyophilization on the formulated CHO-NLB and DSPE-m-PEG-NLB, according to the second and first aspects of the invention respectively, was determined as a function of changes in the average size, zeta potential and DIE of formulations pre- and post-lyophilization and in the absence and presence of a lyoprotectant. Under all conditions, lyophilization appeared to have a destabilizing effect on formulated CHO-NLB. This was more distinctly evident in the resultant zeta potential of formulations following lyophilization, which was markedly less favorable, as highlighted in Table 6 The decrease in surface charge allowed aggregation and coalescence of the NLB, which was evident in the fluctuating average size over the analysis period. Moreover, the ˜9% decrease in DIE observed with CHO-NLB post-lyophilization attested to the instability of the formulation. The structural integrity of the lipid membrane was compromised during the freezing and lyophilization processes leading to reduced incorporation of the lipophilic drug molecule into the NLB-LDDS. The addition of suitable lyoprotectants had an immensely favorable effect on the average size of CHO-NLB determined post-lyophilization. The zeta potential of lyophilized and reconstituted products was comparable to that of pre-lyophilized formulations. The presence of lactose enhanced the DIE of CHO-NLB, demonstrating an insignificant (<2%) decrease relative to that of the DIE achieved prior to lyophilization.

TABLE 6
Tabulation of the physical characteristics of CHO-NLB and DSPE-NLB
formulations highlighting the effect of lyophilization and lyoprotectant
incorporation on the resultant NLB properties.

| | | |
|---|---|---|
| Average | | |
| Size (d · nm) | Zeta Potential (mV) | DIE (%) |

Pre-lyophilization

| | | | |
|---|---|---|---|
| CHO-NLB | 125.60 | −28.10 | 80.10 |
| DSPE-m-PEG-NLB | 84.20 | −7.44 | 59.21 |

Post-lyophilization (no lyoprotectant)

| | | | |
|---|---|---|---|
| CHO-NLB | 278.21 | −22.70 | 71.31 |
| DSPE-m-PEG-NLB | 91.62 | −7.32 | 57.17 |

Post-lyophilization (with lactose)

| | | | |
|---|---|---|---|
| CHO-NLB | 129.40 | −27.90 | 78.65 |
| DSPE-m-PEG-NLB | 85.17 | −8.20 | 57.20 |

By contrast, the effect of lyophilization on DSPE-m-PEG-NLB was distinctly less unfavorable relative to that on CHO-NLB, even in the absence of a lyoprotectant. The presence of the PEG molecule conjugated to DSPE was credited for the stability of this formulation to lyophilization. PEG exhibits cryoprotectant as well as lyoprotectant properties, which facilitated stability of the formulation under freezing and lyophilization conditions. The addition of a lyoprotectant demonstrated comparable size and a marginal improvement in the resultant surface charge characteristics of DSPE-m-PEG-formulations.

The water replacement hypothesis suggests the mechanism of lyoprotection of sugars involves interactions between sugars and the head groups of phospholipids, resulting in maintenance of the spacing of the phospholipid head groups (Chen et al., 2010). Moreover, the sugars also act to reduce the van der Waals forces between the acyl chains of phospholipids, collectively maintaining the structural integrity of the lipid bi-layer membrane.

### 4.3 Determination of Lyoprotectant Efficacy on Formulated NLSs and NLBs

As previously explicated, the process of lyophilization is undertaken to enhance the storage stability of formulations, particularly with regards to NLS. Thorough removal of moisture from the formulation reduces the propensity for hydrolytic degradation and other chemical reactions associated with the presence of water. The maximal water content of lyophilized products deemed acceptable is 3% ^{w}/_{w} (Chaudhury et al., 2012).

In the absence of a lyoprotectant the lyophilized products of CHO-NLS tended to aggregate, requiring slight agitation for loosening. In addition, formulations appeared to be more hygroscopic, showing greater moisture absorption after 48 hours, as was evidenced by the macroscopically observed clumping of the lyophilized powder. Two sugars, fructose and lactose, were investigated for their efficiency as lyoprotectants in the formulations. The presence of fructose in the formulations resulted in a post-lyophilization product that tended to aggregate with a somewhat spongy appearance and texture, particularly in CHO-NLS. Alteration of the concentration of fructose had no significant effect on the texture of the lyophilized product. However, the addition of lactose as a lyoprotectant to CHO-NLS resulted in a more freely flowing powder post-lyophilization. DSPE-m-PEG-NLS showed only very slight aggregation of the lyophilized powder, due to the cryoprotectant and lyoprotectant properties of the PEG molecule. Macroscopic observation of lyophilized DSPE-m-PEG-NLS containing fructose or lactose as lyoprotectants revealed similar, though less pronounced, effects to that observed with CHO-NLS. KF titration corroborated these macroscopic findings, with formulations containing fructose exhibiting approximately 2-4% higher water content on a 10 mg sample size. Lactose-containing NLS samples displayed acceptable water content (<3% ^{w}/_{w}), hence lactose was employed as the lyoprotectant in all ensuing formulations.

### 4.4 SB Incorporation and the Effect of SB on the Physical Characteristics of Formulated NLB

The additional incorporation of silibinin (SB) in the formulated NLB-LDDS was undertaken to enhance the cytotoxic activity of the formulations and provide a means of effective delivery of this poorly soluble phytochemical. However, the maintenance of the nano-scale of the formulation was important and could not be compromised by the addition of a second antineoplastic compound. Moreover, this modification preceded the polymeric coating of the NLB. Hence, only size increments up to 20 nm could be accommodated for CHO-NLB and that of ˜50 nm could be allowed for DSPE-m-PEG-NLB.

The initial incorporation of 100-200 mg of SB resulted in large average sizes and erratic changes in size over time for CHO-NLB. The size distribution was also very broad with PdI>0.6. The size profiles of CHO-NLB obtained following the addition of 15-50 mg SB were notably more favorable. The disparity in the physical characteristics and DIE of CHO-NLB upon the addition of 15 mg and 30 mg SB was marginal. Increasing the quantity of SB to 50 mg resulted in ˜22 nm increase in average of the CHO-NLB, which in turn would not allow for adequate polymeric coating whilst remaining below the about 200 nm benchmark size. Furthermore, the concurrent decrease in surface charge and efficiency of SB incorporation proved unfavorable. The tremendously favorable size profile obtained for DSPE-m-PEG-NLB allowed the LDDS to remain within a suitable size range following the addition of 15-200 mg SB. However, the already unfavorable surface charge was further diminished as the quantity of SB was increased within the defined range, as was the efficiency of SB incorporation. DIE of SB above 50% was identified following the addition of 15 mg and 30 mg SB only. The disparity in average size of DSPE-m-PEG-NLB to which 15 mg and 30 mg SB had been added was insignificant, whilst the zeta potential of the formulations was marginally more favorable following the addition of 30 mg SB. Hence, 30 mg of SB was delineated for incorporation into both CHO-NLB and DSPE-m-PEG-NLB. Table 7 summarizes the resultant average size and zeta potential of CHO-NLB and DSPE-m-PEG-NLB resulting from the addition of a range of SB quantities, as well as the respective efficiency of SB incorporation.

TABLE 7
Physical characteristics of formulated NLB-DDS and efficiency of SB
incorporation relative to quantity of SB added to the formulation

| | CHO-NLB | | | DSPE-m-PEG-NLB | | |
|---|---|---|---|---|---|---|
| | | Zeta | SB | | Zeta | SB |
| SB | Average | Poten- | Incorpo- | Average | Poten- | Incorpo- |
| Added | Size | tial | ration | Size | tial | ration |
| (mg) | (d · nm) | (mV) | (%) | (d · nm) | (mV) | (%) |
| 15 | 137.32 | −27.26 | 66.89 | 91.57 | −8.13 | 54.63 |
| 30 | 137.56 | −27.58 | 65.59 | 93.65 | −8.24 | 52.75 |
| 50 | 159.30 | −21.23 | 61.72 | 93.09 | −7.02 | 43.03 |
| 100 | 246.30 | −18.70 | 50.28 | 99.84 | −6.10 | 36.21 |
| 150 | 249.73 | −18.90 | 46.77 | 132.14 | −6.46 | 30.14 |
| 200 | 267.80 | −16.30 | 43.00 | 143.10 | −6.29 | 24.28 |

### 4.5 Investigating the Feasibility of Polymeric Coating of NLB: Macroscopic and Microscopic Evaluation

The application of successful polymeric coating was assessed through inversion of the zeta potential through positive and negative values following the introduction of an oppositely charged polymer.

The sequential layering of CHO-NLS and DSPE-m-PEG-NLS with chitosan (CHT) and polyacrylic acid (PAA) proved extremely beneficial, displaying adequate inversion of zeta potential following the adsorption of each layer. This change in zeta potential manifested over a shorter period with CHO-NLS. This was attributed to the initially more highly charged nature of the formulated NLS, which resulted in swifter and more complete adsorption of the oppositely charged polymeric layer. Increasing the concentration of PAA from 0.5% ^{w}/ᵥ to 2% ^{w}/ᵥ resulted in an overall strongly anionic surface with no significant polymer precipitation in the suspension. Similarly, decreasing the concentration of CHT from 0.5%% to 0.1%% facilitated the establishment of the desired overall anionic zeta potential. Lyophilization of the NLS following the successful application of four polymeric layers, in the presence of lactose, resulted in a somewhat flaky powder which could be rapidly and easily re-dispersed under ambient conditions. Moreover, the average size of resultant polymer coated-NLB following reconstitution and the introduction of a gaseous core, remained well below about 200 nm (CHO-NLB=189.81 nm; DSPE-m-PEG-NLB=141.62 nm), which was the benchmark size delineated for the stabilized nanosystem. The resultant surface charge of polymer-coated CHO-NLB following the application of four polymeric layers was −32.47 mV and that of DSPE-m-PEG-NLB was −24.27 mV. The institution of polymeric coating had a more pronounced favorable effect on the surface charge of DSPE-m-PEG-NLB than on CHO-NLB. The strongly anionic surface achieved for both formulations had propitious consequences on the stability of NLB formulations. Moreover, anionic surfaces have been reported to have advantageous implications with regard to haemocompatibility and cellular internalization.

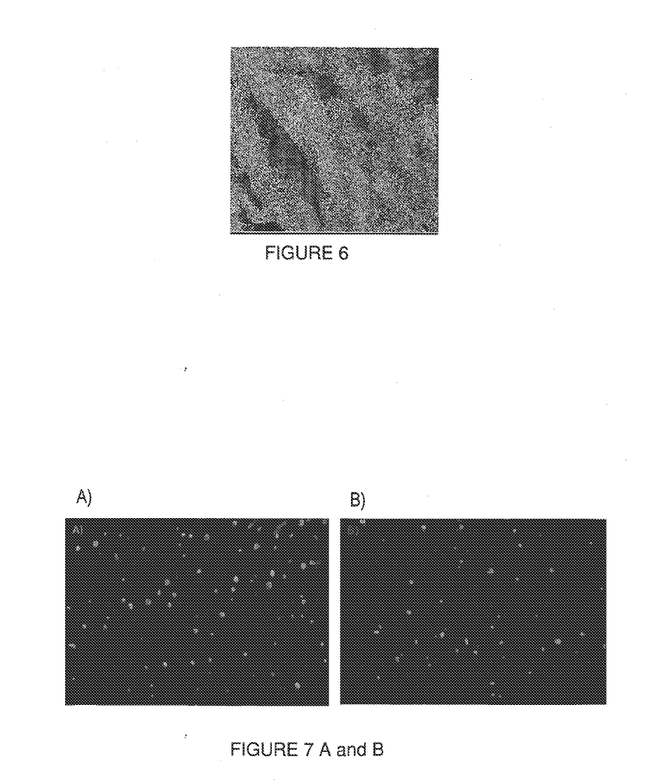

Qualitative assessment of the morphological characteristics of lyophilized layer-by-layer CHT and PAA polymer coated CHO-NLS and DSPE-m-PEG-NLS, employing scanning electron microscopy, provided a deeper understanding of the macroscopic appearance and behavior of the formulations. The micrograph in FIG. 6 of DSPE-m-PEG-NLS coated sequentially with CHT and PAA revealed well defined NLS with the absence of a significant matrix. This substantiates the ease of reconstitution of the samples. The microscopic appearance of CHO-NLS coated with the same polymer combination exhibited comparable characteristics to that observed with coated DSPE-m-PEG-NLS.

### 4.6 Fluorescence Microscopy

Fluorescence microscopy was employed to confirm the restoration of NLS structure and subsequent conversion to NLB, following reconstitution of the lyophilized powder. The fluorescence micrographs of CHO-NLB and DSPE-m-PEG-NLB displayed in FIGS. 7a and b respectively, highlight the resilience of the bi-layer lipid membrane structure and the restoration of the spherical NLB structure. Moreover, there is a distinct absence of aggregation of the NLB.

### 4.7 Establishing the Efficiency of CPT Incorporation

The efficiency of CPT incorporation for candidate CHO-NLS and DSPE-m-PEG-NLS demonstrated exceptionally close correlation to those predicted by statistical optimization, as outlined in Table 8. Conversion of NLS to NLB did not result in significant change in DIE. However, lyophilization followed by reconstitution resulted in ˜2% decrease in DIE for CHO-NLB and DSPE-m-PEG-NLB.

TABLE 8
Experimentally derived and statistically predicted DIE of CPT for
CHO-NLB and DSPE-m-PEG-NLB.

| | DIE | | |
|---|---|---|---|
| Formulation | Experimental (%) | Predicted (%) | Variance (%) |
| CHO-NLB | 81.86 | 82.4128 | 0.007 |
| DSPE-m-PEG-NLB | 59.11 | 59.1864 | 0.001 |

The introduction of SB to the NLB formulations bore the potential to affect all physical and physicochemical characteristics of the formulations, not least of all being CPT incorporation. The considerations, with regards to drug incorporation, following this modification were two-fold. Firstly, the efficiency of SB incorporation was analyzed, since this directly influenced the synergistic antineoplastic effect desired from the introduction of this phytochemical. Secondly, the effect of SB incorporation on the efficiency of CPT incorporation was pertinent to the feasibility of this modification. CPT and SB are both lipophilic compounds and hence were expected to compete for incorporation within the NLB-LDDS. The quantity of SB (30 mg) determined to be the most feasible for incorporation into CHO-NLB and DSPE-m-PEG-NLB displayed >65% incorporation into CHO-NLS with an insignificant decrease in CPT incorporation. DSPE-m-PEG-NLB exhibited a satisfactory incorporation efficiency of SB (˜53%) following the addition of 30 mg SB. However, this formulation repeatedly exhibited a concurrent increase in CPT incorporation of ˜2.5%.

The final modification undertaken on formulated CHO-NLB and DSPE-m-PEG-NLB was the application of sequential layers of polymeric coating. The extended hours required for the complete adsorption of polymeric coats presented a concern with respect to the leakage of drugs from the NLB-LDDS. Coating time was minimized by regular analysis of zeta potential to determine successful coating with the respective polymer in the shortest period. CPT and SB content were assessed before and after the application of polymeric coating.

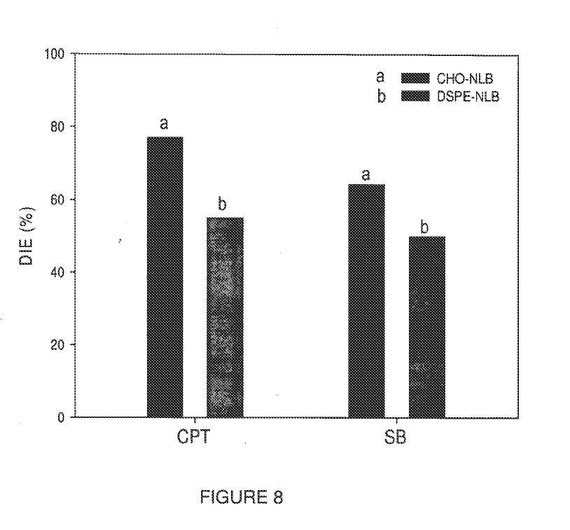

The robustness of CHO-containing bi-layer NLB membranes was again evident by the marginal decrease in CPT and SB content following complete polymeric coating. Moreover, the high surface charge of CHO-NLB perhaps contributed to swifter adsorption of polymers on to the surface, thereby further hindering drug leakage out of the NLB. DSPE-m-PEG-NLB, however, suffered higher drug leakage during the process of polymeric coating. Preliminary studies had displayed more rapid release of drug from DSPE-m-PEG-NLS as opposed to CHO-NLS hence this observation was not entirely unexpected. Nevertheless, CPT content of DSPE-m-PEG-NLB following polymeric coating was only ˜4.5% lower and that of SB was ˜2.7% lower than that achieved prior to the initiation of polymeric coating. FIG. 8 summarizes the final DIE of CPT and SB following polymeric coating, lyophilization and reconstitution in the presence of lactose.

### 4.8 Establishment of Drug Release Characteristics and the Effects of Modifications on Candidate Formulations

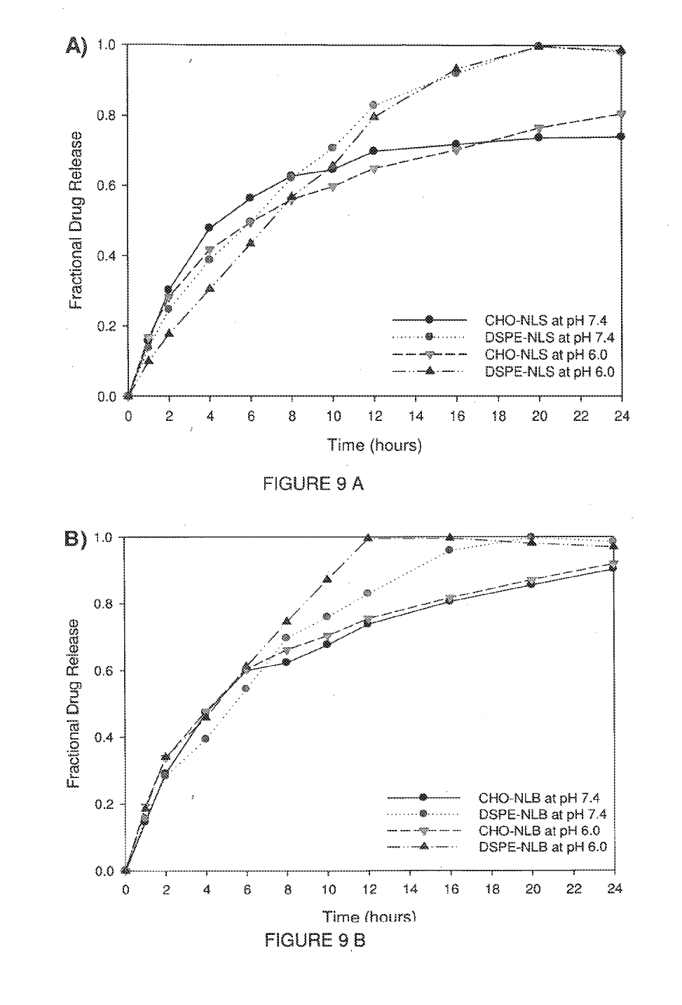

The observed pattern of CPT release from candidate NLS was analogous to the general trend observed with formulations in each of the experimental designs. Candidate NLS and NLB displayed a somewhat bi-phasic CPT release pattern, which was most prominent for CHO-NLS, as illustrated in FIGS. 9a and b. The disparity between release of CPT from CHO-NLS and DSPE-m-PEG-NLS was once again a central feature noted with the candidate NLS. The difference in CPT release may be directly attributed to the average size and surface charge characteristics of each of the candidate NLS. The lower average size of DSPE-m-PEG-NLS provides a greater surface area-to-volume ratio, thereby increasing the area of diffusivity for CPT out of the LDDS. Moreover, the substantially more negative surface charge of CHO-NLS diminishes the tendency for agglomeration of the NLS, thus enhancing the stability of the formulation. CHO-NLS demonstrated a slightly more rapid CPT release than DSPE-m-PEG-NLS over the first 6 hours of analysis. Thereafter the rate of CPT release appeared to decrease. The bi-phasic pattern of DSPE-m-PEG-NLS exhibited faster CPT release for approximately the first 12 hours of the analytical period, followed by a slight decrease in CPT release. Hence the fractional release of CPT from DSPE-m-PEG-NLS exceeds that from CHO-NLS from 10 hours onwards. Complete release of CPT from DSPE-m-PEG-NLS was determined at 20 hours. CHO-NLS displayed only ˜75% cumulative release of incorporated CPT over the 24 hour analysis period. The more acidic pH employed to represent the tumoural environment appeared to have only a marginal effect on CPT release, that varied over the analytical period, from each of the candidate NLS.

The introduction of a gaseous core in the conversion of candidate NLS to NLB resulted in a significantly more rapid release of CPT across all formulations. The first 6 hours of analysis demonstrated very similar CPT release from all of the formulations at physiological and tumoural pH, with the exception of DSPE-m-PEG-NLB at physiological pH. Beyond 8 hours, CPT exhibited a somewhat slower release pattern from CHO-NLB, achieving between 90-92% cumulative CPT release over the 24 hour analytical period under both pH conditions. The significant decrease in surface charge that followed the conversion of CHO-NLS to lyophilized and reconstituted CHO-NLB (−37.9 mV to −27.9 mV) was attributed with the increase in CPT release from the candidate CHO-NLB. A decrease in the intensity of a charged surface results in a less stable formulation that has a greater propensity for aggregation of the NLB. Notwithstanding the decrease in surface charge of CHO-NLB, the zeta potential achieved following conversion to NLB was highly satisfactory, accounting for the absence of a significant burst release from the NLB formulation as well as the controlled pattern of CPT release. Once again, analysis at the lower pH highlighted no significant consequence on the release of CPT from CHO-NLB.

The release of CPT from DSPE-m-PEG-NLB was notably higher than from DSPE-m-PEG-NLS, particularly at lower pH where complete CPT release was observed by 16 hours. The swifter release of CPT from DSPE-m-PEG-NLB can be attributed somewhat to the low surface charge of the formulation. However, it is postulated that the average size of the formulation as well as permeability of the lipid membrane may further contribute to the pattern of CPT release since the zeta potential of post-lyophilization DSPE-m-PEG-NLB is marginally more favourable than that of DSPE-m-PEG-NLS. The considerably faster release of CPT from DSPE-m-PEG-NLB at pH 6.0 may suggest a higher permeability of the lipid membrane to the SF₆ gas in the core of the NLB at lower pH, resulting in swifter release of the incorporated drug.

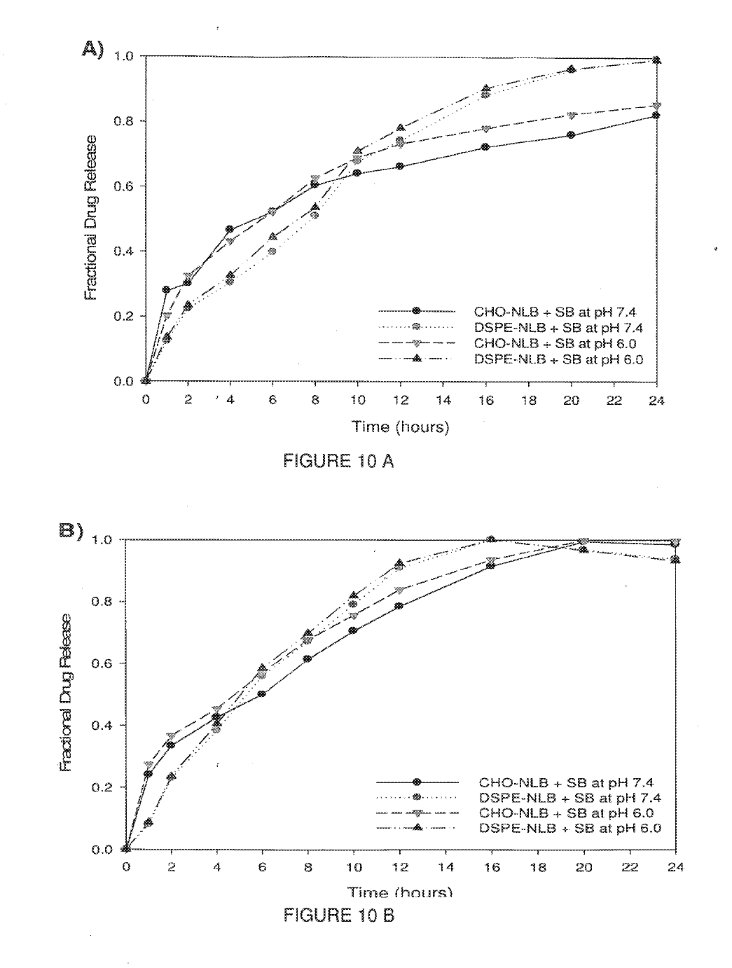

The addition of a second active compound SB, constituted a need to assess the release characteristics of SB as well as determine the effect of SB release on the release profile of CPT. The release pattern of CPT from CHO-NLB containing SB (CHO-NLB+SB) demonstrated no outstanding differences to that of SB naïve formulations for the first 10 hours, except for an evident burst release of CPT over the first hour (as illustrated in FIG. 10a). A similar burst release of SB was observed over this period, displayed in FIG. 10b, suggesting association of both compounds to a certain degree with the surface of the NLB. Furthermore, the presence of an additional compound may have altered the surface tension of the formulated CHO-NLB, leading to the initial burst release of both CPT and SB. From 10 hours the release of CPT from CHO-NLB+SB is approximately 7-9% higher than that observed for CHO-NLB without SB. An effect of the different pH of release medium (7.4 and 6.0) employed during analysis only became evident after 10 hours, when the release of CPT at pH 6.0 appeared to be slightly higher than that at pH 7.4. However, the effect of tumoural pH on the release characteristics of CPT from CHO-NLB+SB was still considered negligible following this study. A cumulative CPT release of 82-86% was achieved for CPT from CHO-NLB+SB over the 24 hour investigation.

The release pattern of CPT from DSPE-m-PEG-NLB containing SB (DSPE-m-PEG-NLB+SB) was slower relative to that for CHO-NLB+SB for the first 10 hours, thereafter exceeding that of CHO-NLB+SB. The lack of significant burst release of CPT and SB suggests association of CPT and SB with the surface of the NLB was absent or to a far lesser extent than suspected for CHO-NLB+SB. The release of CPT from DSPE-m-PEG-NLB+SB was lower than that from SB naïve DSPE-m-PEG-NLB throughout the period under investigation. The difference in pH of the release medium had no demonstrable effect on the release behaviour of CPT from DSPE-NLS+SB. Complete release of CPT from DSPE-m-PEG-NLS+SB was determined by the completion of the 24 hour assessment period. Following the aforementioned burst release of SB from formulated CHO-NLB during the first hour of analysis, the ensuing pattern, exhibited in FIG. 10b, highlights a fairly constant release pattern that is considerably faster than that observed with CPT. SB is more aqueous soluble than CPT which may have contributed to retention of CPT within the lipid environment of the NLB to a greater extent than that of SB. SB appeared to release 3-8% faster at lower pH than at physiological pH over the 24 hour period. Complete release of SB from CHO-NLB was denoted after 20 hours at both physiological and tumoural pH. The pattern of SB release established from DSPE-m-PEG-NLS was remarkably constant, closely resembling first order release. The steeper gradient of the release profile substantiates the achievement of complete SB release from DSPE-m-PEG-NLB+SB in 16 hours. The pH of the release medium had a marginal influence on the release characteristics of SB. The release of SB from DSPE-m-PEG-NLB+SB was significantly more rapid than that of CPT from the same formulation, after the first hour.

The challenge of delivering a poorly aqueous soluble compound that undergoes extensive metabolism has compromised the utilization of SB to its full clinical potential. This phytochemical has, however, demonstrated high permeability in vivo. Incorporation into the NLB-LDDS provides a mechanism of delivery to the tumour tissue where SB can enter tumour cells and exert its anti-neoplastic activity effectively.

The emphasis placed on delaying the onset and reducing the rate of CPT and SB release was established on the need to reduce the indiscriminate systemic activity of these compounds as well as increasing the concentration of CPT and SB at the tumour site. To achieve this, sufficient time was required to allow for passive accumulation of the formulated LDDS at the tumour site before a significant proportion of the incorporated compounds were released. Moreover, extending the release of CPT would be particularly advantageous since the drug acts predominantly in the S-phase of the cell cycle. Hence, extended release facilitates the exposure of a greater quantity of tumour cells in the S-phase to CPT, thereby enhancing the efficacy of CPT. The institution of layered polymeric coating on formulated NLB proved exceptionally advantageous at slowing the release of both CPT and SB from each of the candidate NLB-LDDS.

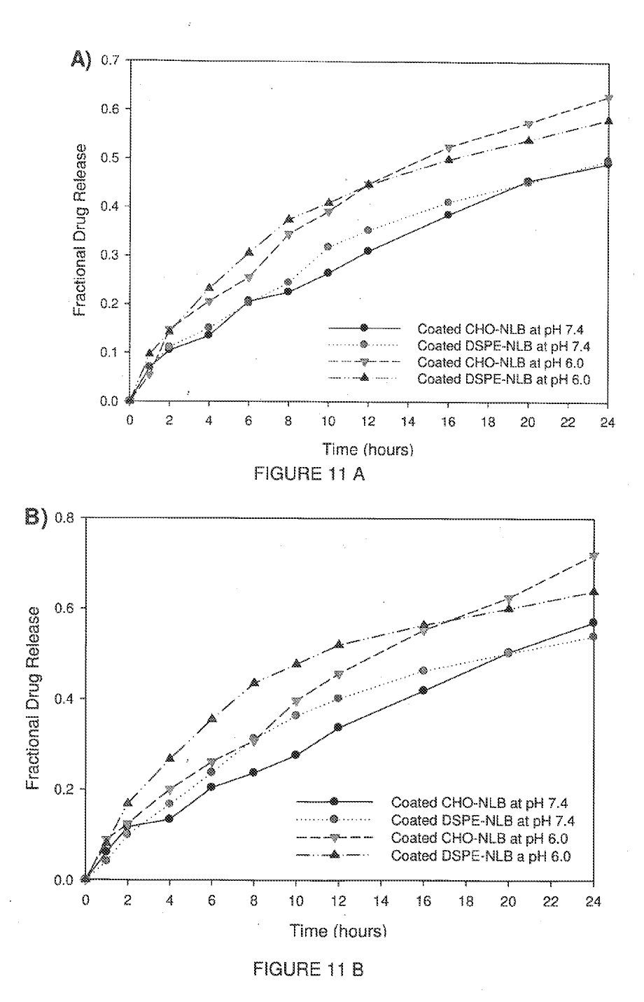

Whilst the application of polymeric coating (in this case layer-by-layer CHT and PAA polymer coating) significantly slowed the release of CPT from CHO-NLB at both physiological and tumoural pH, the disparity in release characteristics was considerably more acute at pH 7.4. The bi-phasic release pattern observed with uncoated NLB was distinctly absent, with the release profile taking on a more constant linear shape, as demonstrated in FIG. 11a. The cumulative release of CPT achieved at 24 hours was <50%. This is a considerable extension of the ≦1 hour half-life of CPT in aqueous medium as reported in the literature (Yang et al., 1999; Kang et al., 2002). In addition, less than 7% of CPT was released from the NLB-LDDS in the first hour of analysis, highlighting the absence of burst release as well as indicating adequately low concentration of CPT released into the systemic circulation. The achievement of the aforementioned release characteristics of CPT from the formulated layer-by-layer CHT and PAA polymer coated CHO-NLB has the potential to address the toxicity profile of CPT which is one of the major drawbacks limiting the clinical application of this broad-spectrum antineoplastic agent.

Evaluation of CPT release at a lower tumoural pH of 6.0, presented in FIG. 11a, also revealed a favorable decrease in the release of CPT over the period of investigation. The release of CPT in the first hour of analysis was lower than that observed for uncoated CHO-NLB at pH 6.0 as well as for layer-by-layer CHT and PAA polymer coated CHO-NLB at physiological pH. Moreover, the release profile of CPT from coated CHO-NLB had a notably more linear appearance than uncoated CHO-NLB, suggesting a more controlled manner of release. However, beyond the first hour the release of CPT from the polymer coated NLB-LDDS was substantially more rapid than observed at physiological pH. A cumulative release of ˜63% CPT was achieved over the 24 hour period, which was more than 14% higher than CPT release attained from coated CHO-NLB over the same period. This was attributed to the pH-responsive property of CHT which was employed as part of the layer-by-layer coating. CHT is a linear polysaccharide that demonstrates aqueous solubility up to pH 6.2, due to the protonation of glucosamine units at this lower pH (Pujana et al., 2012; Chatrabhuti and Chirachanchai, 2013). This alteration in the characteristics of CHT at lower pH facilitates use of this polymer for pH-responsive applications. While the formulated NLB-LDDS cannot be considered strictly pH-responsive, increase in the release of CPT at the lower tumoural pH results in increased concentration of the anti-neoplastic drug within tumour tissue which has the potential to significantly enhance therapeutic efficacy of CPT.

Evaluation of the release characteristics of CPT from layer-by-layer CHT and PAA coated DSPE-m-PEG-NLB bore a strong resemblance to that obtained from layer-by-layer CHT and PAA coated CHO-NLB in both a physiological and tumoural pH release medium. A general trend observed with uncoated DSPE-m-PEG-NLB was more rapid release of active compounds relative to uncoated CHO-NLB. This observation was attributed to lower stability of the DSPE-NLB, due to the less anionic surface charge, as well as the higher permeability of DSPE-NLB lipid membrane. Following layer-by-layer CHT and PAA polymeric coating, the zeta potential of DSPE-m-PEG-NLB demonstrated a tremendously favorable enhancement of the anionic intensity of the surface charge, to a greater extent that the change observed with coated CHO-NLB. In addition, the layer-by-layer CHT and PAA polymeric coating considerably decreased permeability of the LDDS to the SF₆ gaseous core. The coated DSPE-m-PEG-NLB retained some of the bi-phasic release characteristics at pH 6.0 that was discerned from the uncoated formulations. Release of CPT from DSPE-m-PEG-NLB was faster over the first 8 hours of evaluation. The cumulative release of CPT demonstrated from DSPE-m-PEG-NLB at physiological and tumoural pH approximated 50% and 58%, respectively. This favorable release pattern combined with the smaller size of coated DSPE-m-PEG-NLB, relative to that of coated CHO-NLB may prove vastly advantageous to the passive targeting capacity of this LDDS.

As described for CPT, the release of SB from CHO-NLB and DSPE-m-PEG-NLB was significantly reduced as a consequence of the layer-by-layer CHT and PAA polymeric coating, as depicted in FIG. 11b. The burst release of SB from uncoated CHO-NLB was a particular concern. Layer-by-layer CHT and PAA polymeric coating successfully reduced the release of SB from CHO-NLB by ˜19% during the first hour under both pH environments. The influence of pH was tangible, particularly after the first 2 hours of analysis. The release of SB CHO-NLB was slightly faster than that derived for CPT. The cumulative release achieved for SB from CHO-NLB at pH 7.4 and 6.0 were 57% and ˜72%, respectively. Investigation of DSPE-m-PEG-NLB also highlighted considerably slower release of SB following layer-by-layer CHT and PAA polymeric coating of the LDDS. There was a pronounced influence of pH on the release of SB from coated DSPE-m-PEG-NLB, with cumulative release at physiological pH˜10% lower than that determined at tumoural pH. The reduced release of SB at physiological pH indicates a lower concentration of SB will be subjected to metabolism in the systemic circulation and clearance prior to reaching the tumour tissue. The quicker release of SB from coated DSPE-m-PEG-NLB at tumoural pH will facilitate the achievement of higher concentrations of SB at the target site, resulting in superior SB efficacy as well as enhanced synergistic antineoplastic effect with CPT.

### 4.9 Defining the Stability Characteristics of NLB-LDDS

Stability of pharmaceutical formulations can significantly influence viability of the formulation from cost, production and clinical use standpoints. Formulations that cannot be stored for an acceptable period require production shortly before use which can result in an increase in production and transportation costs, delays in treatment due to unforeseen circumstances and ultimately complicate clinical use. Moreover, post-reconstitution time-dependent stability of lyophilized products, suitable storage conditions, as well as post-administration stability is pivotal to the assessment of the overall feasibility of formulations.

### 4.9.1 Determination of the Stability of NLB in Serum

The intended intravenous delivery of the NLB formulations demands the establishment of stringent stability parameters, particularly with regards to the size characteristics of administered formulations. The adsorption of serum proteins, or the aggregation of NLB in the presence of serum proteins can significantly affect the feasibility of the formulation. Uncoated CHO-NLB exhibited a <10 nm increase in size in the presence of serum proteins, over the analysis period, as well as a marginal decrease in surface charge. This was attributed to slight destabilization of the CHO-NLB in the presence of serum proteins which resulted in aggregation of the NLB. The increase in surface charge following layer-by-layer CHT and PAA polymer coating of the CHO-NLB afforded greater stability to the formulation. Hence the presence of serum proteins had only a marginal effect on the stability of the formulation. The <2 nm increase in size was not attributed to the presence of serum proteins, but rather to normal size variation of the nanosystem over time. Maintenance of the zeta potential was a further indication that aggregation of the CHO-NLB was absent.

TABLE 9
Physical characteristics of uncoated and layer-
by-layer CHT and PAA polymer coated CHO-NLB
and DSPE-m-PEG-NLB in the presence of FBS

| | Uncoated NLB | | | Polymer-coated NLB | | |
|---|---|---|---|---|---|---|
| | Average | | Zeta | Average | | Zeta |
| Time | Size | | Potential | Size | | Potential |
| (min) | (d · nm) | PdI | (mV) | (d · nm) | PdI | (mV) |

CHO-NLB

| | | | | | | |
|---|---|---|---|---|---|---|
| 0 | 137.56 | 0.19 | −27.58 | 189.81 | 0.21 | −32.47 |
| 15 | 140.02 | 0.23 | −25.83 | 190.01 | 0.20 | −32.41 |
| 30 | 140.67 | 0.27 | −24.02 | 190.72 | 0.23 | −31.98 |
| 45 | 143.91 | 0.24 | −24.78 | 191.06 | 0.19 | −32.42 |
| 60 | 145.32 | 0.29 | −23.35 | 191.39 | 0.21 | −31.77 |

DSPE-m-PEG-NLB

| | | | | | | |
|---|---|---|---|---|---|---|
| 0 | 93.65 | 0.26 | −8.24 | 141.62 | 0.21 | −24.27 |
| 15 | 93.91 | 0.22 | −8.20 | 141.84 | 0.19 | −24.21 |
| 30 | 93.86 | 0.21 | −7.94 | 141.91 | 0.20 | −24.00 |
| 45 | 94.27 | 0.24 | −8.17 | 142.06 | 0.21 | −23.84 |
| 60 | 94.91 | 0.21 | −8.21 | 142.24 | 0.21 | −23.87 |

There was a distinct absence of membrane destabilization for uncoated and layer-by-layer CHT and PAA polymer-coated DSPE-m-PEG-NLB in the presence of serum proteins, as evidenced by the minute variations in size and zeta potential of formulations of the analysis period. The presence of PEG conjugated to DSPE in the membrane of the formulated DSPE-m-PEG-NLB conferred superior stability to the formulation against the effects of serum proteins. The strong anionic charge of polymer-coated DSPE-m-PEG-NLB was accredited for the stability of the formulation against aggregation and interaction with serum proteins.

### 4.9.2 Characterizing the Stability of Reconstituted NLB

The reconstitution of lyophilized powders or particulate formulations into suspensions is accompanied by a change in the stability of the formulation. Preparation and administration instructions by manufacturers of some cytotoxic preparations define a period of just 4-6 hours between reconstitution of the product and complete intravenous infusion of the cytotoxic preparation. The stability of formulated NLB-LDDS was assessed at ambient temperature employing a Turbiscan™ LAB (Formulaction, L'Union, France). Determination of the light backscattered by the layer-by-layer CHT and PAA coated and uncoated NLB preparations was employed to define stability characteristics of the formulations. The Turbiscan™ LAB is able to detect minute alterations in the behavior of suspended matter considerably earlier than macroscopic observation will allow.

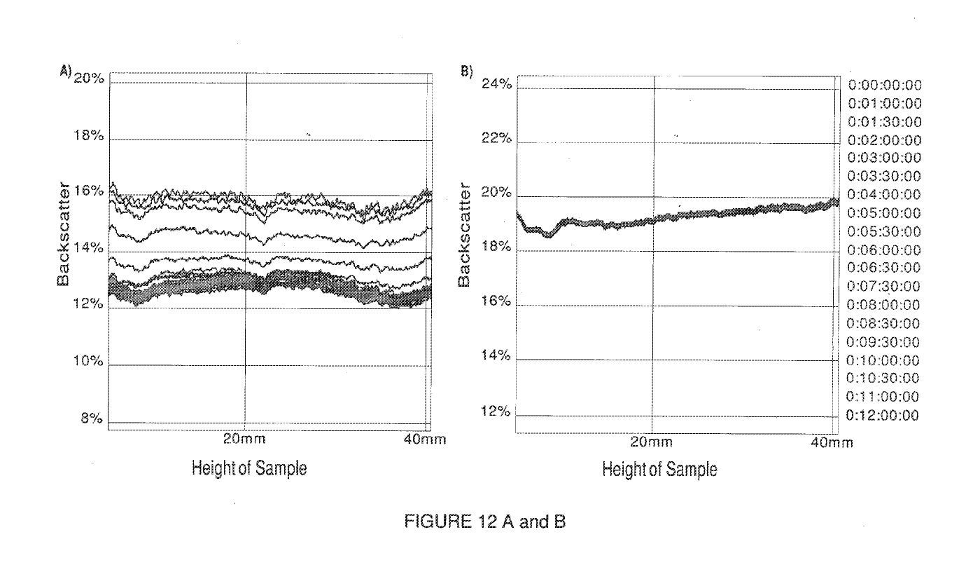

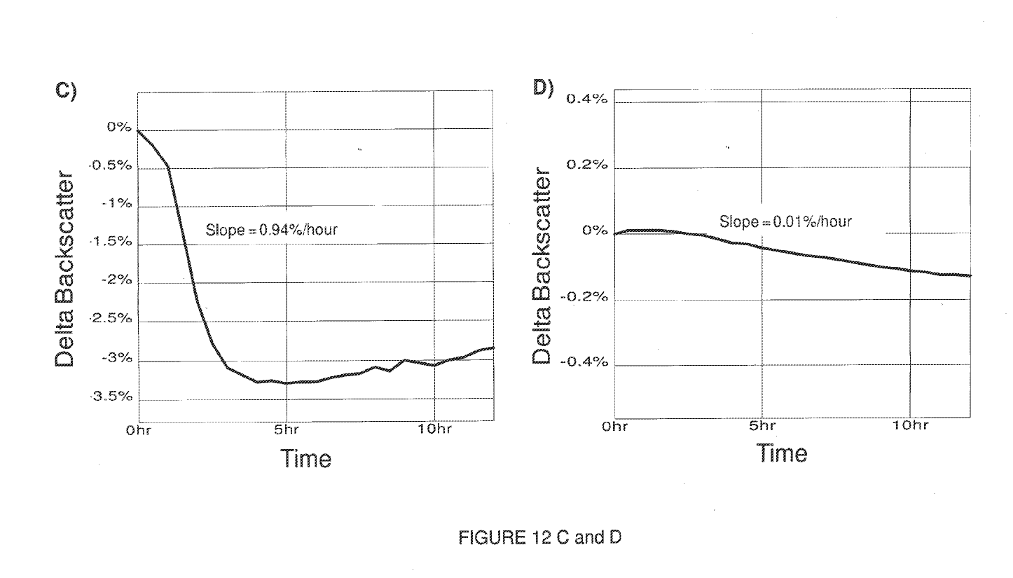

The backscatter plot of uncoated CHO-NLB (depicted in FIG. 12a) highlighted no localized changes in the behavior of the particulate matter, which would be indicative of sedimentation or creaming of the suspended NLB. However, the change in backscatter across the entire spectrum suggested a change in the size of the NLB. A decrease in size was observed over the first 6 hours post reconstitution, which may have been the result of gradual evaporation of the gaseous core out of the NLB. Thereafter a marginal increase (<2%) in size of the CHO-NLB was observed. The decrease in formulation stability that is associated with time after reconstitution resulted in aggregation, and possibly coalescence, of CHO-NLB thereby causing the increase in size detected by the increase in backscatter. The maximal variation in backscatter determined over the 12 hour period following reconstitution approximated 4%. The change in backscatter was quantified over the analytical period and is depicted in FIG. 12c. The most distinct change in backscatter, as indicated by the steepest gradient, was observed from 0.5-4.5 hours with a 0.94%/hour change in backscatter.

The backscatter profile for layer-by-layer CHT and PAA polymer coated CHO-NLB, depicted in FIG. 12b represents an exemplary display of formulation stability. Similar to the uncoated CHO-NLB, there was no evidence of creaming or sedimentation. A marginal (<2%) variation in size of the CHO-NLB was observed over the 12 hour period. However, unlike with uncoated CHO-NLB, only a uni-directional size variation was observed. The slight reduction in size of layer-by-layer CHT and PAA coated CHO-NLB was attributed to gradual permeation of SF₆ gas out of the NLB. The enhanced stability of the formulated layer-by-layer CHT and PAA coated CHO-NLB underlies the absence of aggregation that was observed with the uncoated CHO-NLB from 6 hours post-reconstitution. FIG. 12d quantifies the change in backscatter referenced to the initial measurement, over the analytical period. The gradient of the graph highlights the exceptional stability of the polymer coated CHO-NLB formulation with a 0.01% change in backscatter per hour.

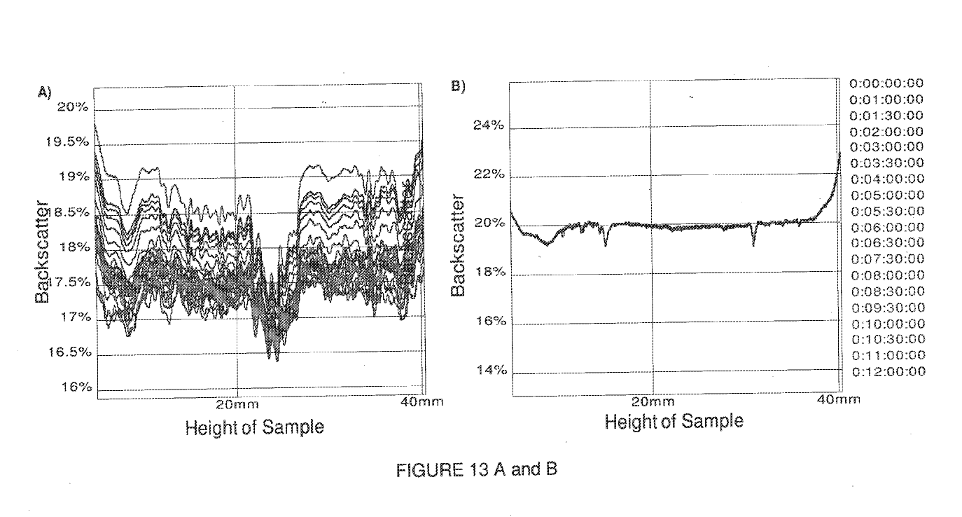

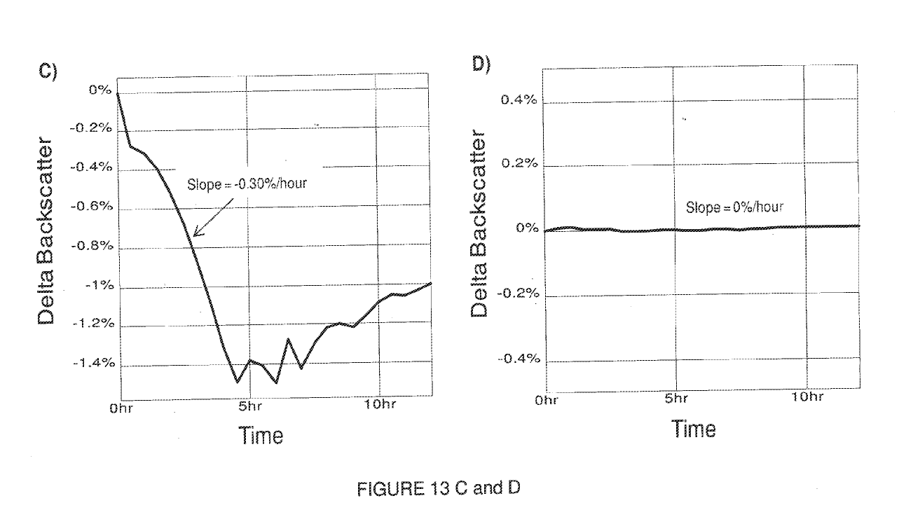

There is a definitive absence of migrational behavior of uncoated and layer-by-layer CHT and PAA coated DSPE-m-PEG-NLB, confirming stability of the formulation against sedimentation or creaming. The backscatter plot of uncoated DSPE-m-PEG-NLB, presented in non-referenced mode in FIG. 13a, highlights comparable characteristics to uncoated CHO-NLS with regards to a reduction in NLB size observed during the first half of the analysis period, followed by an increase in size of the suspended NLB. This phenomenon was again attributed to the evaporation of the gaseous core from the DSPE-m-PEG-NLB resulting in diminished size of the LDDS followed by aggregation of the less stable DSPE-m-PEG-NLB during the latter stages of analysis. The maximal change in backscatter observed over the 12 hour period approximated 2.5%. Whilst DSPE-m-PEG-NLB were less stable than CHO-NLB, as concluded by the zeta potential displayed by each formulation, the change in backscatter was less than the 4% variation observed for CHO-NLB. The smaller initial size of uncoated DSPE-m-PEG-NLB contained a smaller volume of SF₆ gas in the core of the formulation. Consequently, evaporation of the gaseous core out of the formulated DSPE-m-PEG-NLB caused a slighter size variation than that observed with larger CHO-NLB. The change in backscatter was quantified by means of the DeltaBS(t) plot depicted in FIG. 13c. This plot highlights the largest reduction in backscatter over the 0.5-4.5 hours, followed by alternating increase and decrease in backscatter which suggests insignificant size variation due to reversible aggregation of the suspended NLB in Brownian motion. The slope of the graph between 0.5-4.5 hours indicates a 0.3% change in backscatter per hour.

The backscatter plot of polymer coated DSPE-m-PEG-NLB presented a significantly more favorable scenario with regards to stability characteristics of the LDDS. This graph, presented in non-referenced mode in FIG. 13b, highlighted an almost inconceivable change in backscatter over the 12 hour analysis period. This exceptional stability was confirmed by quantification of the change in backscatter with reference to the initial measurement at the start of the assessment, presented in FIG. 13d. The virtually horizontal gradient of the graph concludes a 0% change in backscatter per hour over the entire 12 hour period. Hence, the stability profile of DSPE-m-PEG-NLB following reconstitution suggests a highly stable formulation that will allow sufficient time between reconstitution and administration to patients.

### 4.9.3 Evaluation of the Storage Stability of the NLB-LDDS

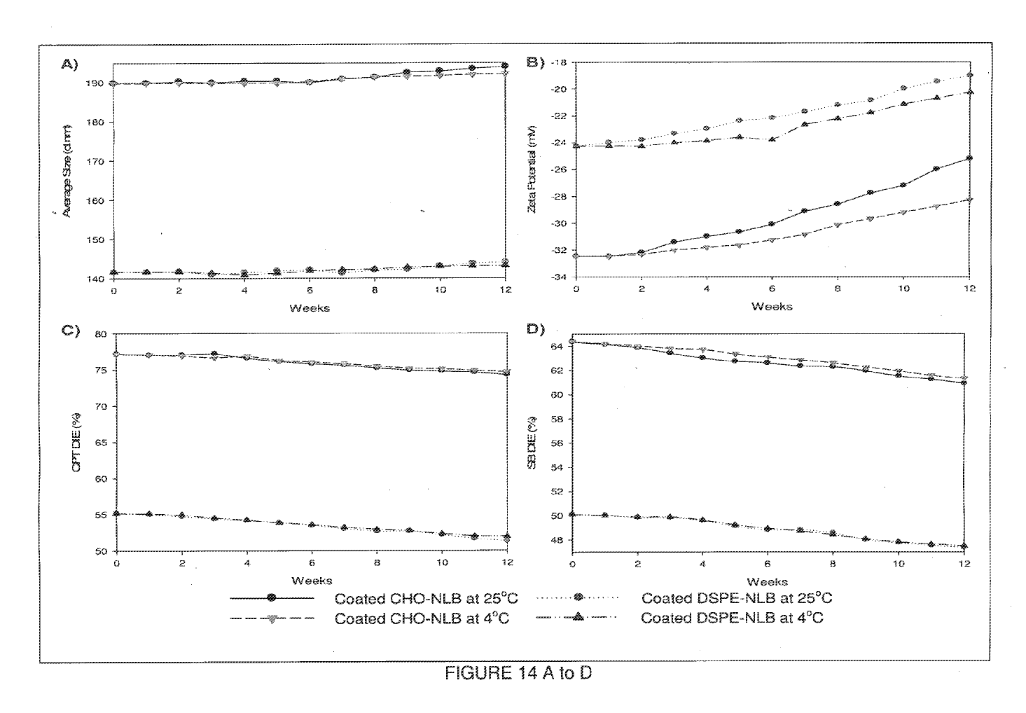

The stability of formulated layer-by-layer CHT and PAA polymer coated CHO-NLB and DSPE-m-PEG-NLB as a lyophilized product was assessed over a 3 month period under ambient and refrigeration temperatures. At weekly intervals the formulations were reconstituted, converted to NLB and the average size, zeta potential of the formulations as well as DIE of both CPT and SB were determined. The change in size of CHO-NLB was minimal over the first 8 weeks, following which refrigerated formulations maintained their size better than formulations stored at room temperature, as highlighted in FIG. 14a. However, the difference between refrigerated and non-refrigerated formulations was <2 nm during the third month. At the conclusion of the 12 week study, the CHO-NLB formulations remained below 200 nm. DSPE-m-PEG-NLB displayed an insignificant variation in the average size of formulations stored at both temperatures over the entire study. In addition, the formulations exhibited only a 2-3 nm increase in size by the end of the investigation. Long term storage, as well as the storage temperatures, appeared to have a greater impact on the surface charge of formulations, as illustrated in FIG. 14b. CHO-NLB stored at ambient temperature demonstrated an unfavorable 7.24 mV increase in zeta potential over the assessment period, whilst refrigerated samples bore a 4.17 mV increase in zeta potential. The disparity in zeta potential between refrigerated and non-refrigerated samples increased as the study proceeded.

The leakage of incorporated drugs out of formulated liposomes and nanobubbles stored in suspension presents one of the fundamental stability challenges related to the feasibility of these LDDS. Lyophilization has been previously explored and reported as a means of enhancing the storage stability of the aforementioned LDDS. Hence, the final presentation of CHO-NLB and DSPE-m-PEG-NLB formulated in this study was lyophilized products. The storage stability of both formulations with regards to the DIE of CPT over the analytical period, presented in FIG. 14c, was outstanding. CHO-NLB displayed <3% decrease in the DIE of CPT, whilst the decrease was <4% for DSPE-m-PEG-NLB by the conclusion of the analysis. The influence of storage temperature on the incorporation of CPT in both DDS was insignificant. Storage temperature, as depicted in FIG. 14d, appeared to have a negligible impact on the efficiency of SB incorporation in DSPE-m-PEG-NLB. However, storage time resulted in a gradual decrease in SB incorporation, culminating in a <3% lower DIE for SB at the end of the 3 month study. The storage stability of CHO-NLB with respect to SB incorporation was slightly less favorable than observed with DSPE-m-PEG-NLB, with FIG. 14d denoting ˜4% decrease in SB DIE over the period of investigation. Between weeks 3-8, the influence of refrigeration on the incorporation of SB is evident. However, overall the influence of storage temperature on the incorporation stability of SB was negligible for CHO-NLB.

### 4.10 Advantages for the NLB-LDDSs According to the Invention

Conversion of the NLS to the NLB formulation highlighted superior stability, drug incorporation and drug release characteristics of CHO-NLB, whilst the size profile of DSPE-m-PEG-NLB was particularly favorable. The feasibility of lyophilization was investigated as a practical consideration to improve the long term stability of the formulation, thereby enhancing the industrial and clinical viability of the novel LDDSs. Fluorescence microscopy further confirmed the restoration of the morphological structure of NLB following lyophilization.

Further modifications undertaken on the formulated NLB-LDDS included the incorporation of a phytochemical with antineoplastic properties. Maintaining a favorable size profile to facilitate the passively targeted nature of the LDDS was a central consideration in determining the optimal concentration of SB to be incorporated into the LDDS. Interestingly the incorporation of SB into CHO-NLB had a marginal effect on the concurrent DIE of CPT and a slight increase in CPT incorporation into DSPE-m-PEG-NLB. The inclusion of SB into the DDS resulted in a burst release of both CPT and SB from CHO-NLB during the first hour, as well as more rapid release of CPT over the 24 hour assessment period. By contrast, the incorporation of SB into DSPE-m-PEG-NLB resulted in a slower release of CPT. The higher aqueous solubility of SB relative to CPT was attributed with the more rapid release of SB, with complete release of SB being achieved in 20 hours for CHO-NLB and 16 hours for DSPE-m-PEG-NLB.

In terms of polymeric layering, the combination of CHT and PAA proved immensely favorable, with lyophilization producing a flaky powder that was easily reconstituted. In addition, the average size of both CHO- and DSPE-m-PEG-NLB remained below the benchmark 200 nm size initially outlined. The bearing of polymeric coating had a particularly advantageous impact on the surface charge of DSPE-m-PEG-NLB, with a favorable ˜16 mV decrease in zeta potential. CHO-NLB displayed incorporation efficiencies of 77.17% and 64.38% respectively for CPT and SB. DSPE-m-PEG-NLB displayed incorporation efficiencies of 55.17% and 50.10%, respectively, for CPT and SB. Moreover, the application of polymeric coating significantly enhanced the release characteristics of both drug compounds and introduced differential release characteristics at physiological and tumoural pH, thereby improving the passive targeting capacity of the LDDSs.

Stability of formulations is a pivotal consideration for pharmaceutical formulations. The assessment of stability of the formulated NLB-LDDS further highlighted the impact of polymeric coating (particularly layer-by-layer CHT and PAA polymeric coating) on the stability characteristics of CHO-NLB and DSPE-m-PEG-NLB. Post-reconstitution evaluation of polymer coated CHO-NLB and DSPE-m-PEG-NLB denoted remarkable stability characteristics for the entire 12 hour assessment period, particularly for DSPE-m-PEG-NLB. The evaluation of long term storage stability of the NLB-LDDS under ambient and refrigerated temperatures over a 3 month period highlighted excellent stability with regards to the incorporation of CPT and SB with insignificant influence of storage temperature. The increase in size of CHO-NLB was only evident after 8 weeks whilst the non-refrigerated formulation underwent an unfavorable >7 mV increase in zeta potential. The size and zeta potential profiles of DSPE-m-PEG-NLB over the three month assessment period demonstrated superior stability.

The CHO-NLS, CHO-NLB, DSPE-m-PEG-NLS and DSPE-m-PEG all in accordance with this invention provide for drug delivery systems that at least have favourable sizes for passive tumoural targeting, are stable for storage purposes, are readily formulated into intravenous chemotherapy applications, show favourable drug incorporation efficiencies, and show favourable drug release profiles. The LDDSs presented herein each at least alleviates a known problem in the current state of the art.

While the invention has been described in detail with respect to specific embodiments and/or examples thereof, it will be appreciated that those skilled in the art, upon attaining an understand of the foregoing may readily conceive of alterations to, variations of and equivalents to these embodiments. Accordingly, the scope of the present invention should be assessed as that of the appended claims and any equivalents thereto.

### REFERENCES

1. Chen K-I, Li B-R, Chen Y-T. Silicon nanowire field-effect transistor-based biosensors for biomedical diagnosis and cellular recording investigation, Nano today, (2011); 6(2): 131-154.
2. Chien J R, Aletti G, Bell D A, Keeney G L, Shridhar V, Hartmann L C. Molecular pathogenesis and therapeutic targets in epithelial ovarian cancer, Journal of Cellular Biochemistry, (2007), 102 (5): 1117-1129.
3. Cho Y W, Park S A, Han T H, Son D H, Park J S, Oh S J, Moon D H, Cho K-J, Ahn C-H, Byun Y, Kim I-S, Kwon I C, Kim S Y. In vivo tumour targeting and radionuclide imaging with self-assembled nanoparticles: Mechanisms, key factors, and their implications, Biomaterials, (2007); 28(6): 1236-1247.
4. Cirstoiu-Hapca A, Buchegger F, Lange N, Gurny R, Delie F. Benefit of anti-HER2-coated paclitaxel-loaded immune-nanoparticles in the treatment of disseminated ovarian cancer: Therapeutic efficacy and biodistribution in mice, Journal of Controlled Release, (2010); 144(3): 324-331.
5. Dominguez A L, Lustgarten J. Targeting the tumour microenvironment with anti-neu/anti-CD40 conjugated nanoparticles for the induction of antitumor immune responses, Vaccine, (2010); 28(5): 1383-1390.
6. Fan N, Duan K, Wang C, Liu S, Luo S, Yu J. Fabrication of nanomicelle with enhanced solubility and stability of camptothecin based on α,β-poly[(N-carboxybutyl)-L-aspartamide]-camptothecin conjugate, Colloids and Surfaces B: Interfaces, (2010); 75(2): 543-549.
7. Ferrandina G, Legge F, Salutari V, Paglia A, Testa A, Scambia G. Impact of pattern of recurrence on clinical outcome of ovarian cancer patients: Clinical considerations, European Journal of Cancer, (2006); 24(14): 2296-2302.
8. Guo M, Que C, Wang C, Liu X, Yan H, Liu K. Multifunctional superparamagnetic nanocarriers with folate-mediated and pH-responsive targeting properties for anticancer drug delivery, Biomaterials, (2011); 32 (1): 185-194.
9. Jiu J, Jiang Z, Zhang S, Saltzman W M. Poly(ω-pentadecalactone-co-butylene-co-succinate) nanoparticles as biodegradable carriers for camptothecin delivery, Biomaterials, (2009); 30(29): 5707-5719.
10. Khosravi-Darani K, Pardakhty A, Honarpisheh H, Rao V S N M, Mozafari M R. The role of high-resolution imaging in the evaluation of nanosystems for bioactive encapsulation and targeted nanotherapy, Micron, (2007); 38(8): 804-818.
11. Kim P S, Djazayeri S, Zeineldin R. Novel nanotechnology approaches to diagnosis and therapy of ovarian cancer, Gynecologic Oncology, (2011), 120(3): 393-403.
12. Knopp D, Tang D, Niessner R, Review: Bioanalytical applications of biomolecule-functionalized nanometer-sized doped silica particles. Analytica Chimica Acta., (2009); 647 (1): 14-30.
13. Mohanty C, Sahoo S K. The in-vitro stability and in-vivo pharmacokinetics of curcumin prepared as an aqueous nanoparticulate formulation, Biomaterials, (2010); 31(25): 6597-6611.
14. Park J W. Liposome-based drug delivery in breast cancer treatment, Breast Cancer Research, (2002); 4(3): 95-99.
15. Ranjan R, Vaidya S, Thaplyal P, Qamar M, Ahmed J and Ganguli A K. Controlling the size, morphology and aspect ratio of nanostructures using reverse micelles: A case study of copper oxalate monohydrate. Langmuir, (2009); 25 (11): 6469-6475.
16. Schluep T, Cheng J, Khin K T, Davis M E. Pharmacokinetics and biodistribution of the camptothecin-polymer conjugate IT-101 in rats and tumour-bearing mice, Cancer Chemotherapy and Pharmacology, (2006); 57: 654-662.
17. Shapira A, Livney Y D, Broxterman H J, Assaraf Y G. Nanomedicine for targeted cancer therapy: towards the overcoming of drug resistance, Drug Resistance Updates, (2011); 14(3): 150-163.
18. Shen Y, Tang H, Zhan Y, Van Kirk E A, Murdoch W J. Degradable Poly(β-amino ester) nanoparticles for cancer cytoplasmic drug delivery, Nanomedicine, (2009); 5(2): 192-201.
19. Stuart G C E. First-line treatment regimens and the role of consolidation therapy in advanced ovarian cancer, Gynecologic Oncology, (2003); 90(3): S8-S15.
20. Tang J, Rangayyan R, Yao J, Yang Y. Digital image processing and pattern recognition techniques for the detection of cancer, Pattern Recognition, (2009); 42: 1015-1016.
21. Vauthier C, Dubernet C, Chauvierre C, Brigger I, Couvreur P. Drug delivery to resistant tumours: the potential of poly(alkyl cyanoacrylate) nanoparticles, Journal of Controlled Release, (2003); 93(2): 151-160.
22. Vizirianakis I S, Nanomedicine and personalized medicine toward the application of pharmacotyping in clinical practice to improve drug-delivery outcomes, Nanomedicine: Nanotechnology, Biology and Medicine, (2011); 7(1): 11-17.

1. A liposomal drug delivery system (LDDS) for the release of at least one drug compound to a target site in a human or animal body, the liposomal drug delivery system (LDDS) comprising a liposomal shell consisting of distearoyl phosphocholine (DSPC) and distearoyl phosphatidylethanolamine-m-polyethylene glycol (DSPE-m-PEG), the shell defining an inner compartment.

2. The liposomal drug delivery system (LDDS) according to claim 1, wherein the liposomal shell further comprises a surfactant.

3. The liposomal drug delivery system (LDDS) according to claim 2, wherein the surfactant is at least one surfactant selected from the group consisting of: dioctyl sulfosuccinate (DOS), Tween 80 and Span 80, or any combination thereof.

4. The liposomal drug delivery system (LDDS) according to claim 1, further comprising a polymeric coating at least partially covering the shell.

5. The liposomal drug delivery system (LDDS) according to claim 4, wherein the polymeric coating is at least one polymeric coating selected from the group consisting of: biocompatible polymers, ionic polymers, anionic, cationic polymers, gelatin, polyethyleneimine (PEI), poly-L-lysine (PLL), carrageenan, pectin, sodium alginate, carboxylic, sulfate, and amine functionalized polymers such as polyacrylic acid (PAA), polymethacrylic acid, polyethylene amine, polysaccharides such as alginic acid, pectinic acid, carboxy methyl cellulose, hyaluronic acid, heparin (mucopolysaccharide), chitosan (CHT), carboxymethyl chitosan, carboxymethyl starch, carboxymethyl dextran, heparin sulfate, chondroitin sulfate, cationic guar, cationic starch, and their salts, poly (butyl cyanoacrylate) (PBCA), poly(lactic acid) (PLA), poly(propylene fumarate)(PPF), polyanhydrides.

6. The liposomal drug delivery system (LDDS) according to claim 5, wherein the polymeric coating comprises at least two layers, preferably an anionic layer and a cationic layer, further preferably polyacrylic acid (PAA) and chitosan (CHT).

7. The liposomal drug delivery system (LDDS) according to claim 1, wherein the liposomal shell further comprises a lyoprotectant, preferably a sugar, further preferably lactose and/or fructose.

8. The liposomal drug delivery system (LDDS) according to claim 1, further comprising a gas housed within the inner compartment defined by the shell so as to form a nanolipobubble (NLB).

9. The liposomal drug delivery system (LDDS) according to claim 8, wherein the gas is at least one gas selected from the group consisting of, air, nitrogen, oxygen, carbon dioxide, hydrogen, nitrous oxide, a noble or inert gas such as helium, argon, xenon or krypton; a radioactive gas such as Xe¹³³ or Kr⁸¹; a hyperpolarized noble gas, a low molecular weight hydrocarbon such as methane, ethane, propane, butane, isobutane, pentane or isopentane; a cycloalkane such as cyclobutane or cyclopentane; an alkene such as propene, butene or isobutene; or an alkyne such as acetylene; an ether; a ketone; an ester; halogenated gases, preferably fluorinated or perfluorinated gases, such as fluorinated hydrocarbons; sulphur hexafluoride; perfluoroacetone; perfluorodiethyl ether; perfluoroalkanes; perfluoroalkenes; perfluoroalkynes; perfluorocycloalkanes; and saturated perfluorocarbons, preferably, the gas is sulphur-hexa fluoride

10. The liposomal drug delivery system (LDDS) according to claim 1, further comprising a drug compound housed inside the inner compartment defined by the liposomal shell.

11. The liposomal drug delivery system (LDDS) according claim 10, wherein the drug compound is at least one drug selected from the group consisting of: amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, antiparkinson agents, antidepressants, anti-neoplastic drugs, immunosuppressants, antiviral agents, antibiotic agents, antifungal agents, antimicrobial agents, appetite suppressants, antiemetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds; oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol-lowering agents, phytochemicals, and antioxidants, or any derivative of the aforementioned.

12. The liposomal drug delivery system (LDDS) according to claim 11, wherein the anti-neoplastic drug is at least one anti-neoplastic drug selected from the group consisting of: camptothecin, taxanes and platinum compounds.

13. The liposomal drug delivery system (LDDS) according to claim 1, wherein the distearoyl phosphocholine (DSPC) is 1,2-distearoyl-sn-glycero-3-phosphocholine.

14. The liposomal drug delivery system (LDDS) according to claim 1, wherein the distearoyl phosphatidylethanolamine-m-polyethylene glyclol (DSPE-m-PEG) is L-α-distearoylphosphatidylethanolamine-methoxy-polyethylene glycol conjugate (DSPE-m-PEG).

15. The liposomal drug delivery system (LDDS) according to claim 1, wherein the liposomal shell is configured such that non-polar functional groups of the distearoyl phosphocholine (DSPC) and the distearoyl phosphatidylethanolamine-m-polyethylene glycol (DSPE-m-PEG) are directed inwardly toward the inner compartment and polar functional groups are directed outwardly toward an outer surface of the shell, in use, the non-polar functional groups of the liposomal shell increases the solubilisation of non-polar and/or lipophilic drug compounds housed within the compartment.

16. A liposomal drug delivery system (LDDS) for the release of at least one drug compound to a target site in a human or animal body, the liposomal drug delivery system (LDDS) comprising:
a nanoliposomal shell consisting of distearoyl phosphocholine (DSPC), distearoyl phosphatidylethanolamine-m-polyethylene glycol (DSPE-m-PEG) and a surfactant, the shell defining an inner compartment; and
a drug compound housed inside the inner compartment defined by the nanoliposomal shell.

17. The liposomal drug delivery system (LDDS) according to claim 16, wherein the surfactant is dioctyl sulfosuccinate (DOS).

18. The liposomal drug delivery system (LDDS) according to claim 16, wherein the nanoliposomal shell further comprises a polymeric coating at least partially covering the shell, preferably the polymeric coating comprises at least two layers, preferably an anionic layer and a cationic layer, further preferably polyacrylic acid (PAA) and chitosan (CHT).

19. The liposomal drug delivery system (LDDS) according to claim 16, wherein the nanoliposomal shell further comprises a lyoprotectant, preferably a sugar, further preferably lactose and/or fructose.

20. The liposomal drug delivery system (LDDS) according to claim 16, further comprising a gas housed within the inner compartment so as to form a nanolipobubble (NLB).

21. A liposomal drug delivery system (LDDS) for the release of at least one drug compound to a target site in a human or animal body, the liposomal drug delivery system (LDDS) comprising a liposomal shell consisting of distearoyl phosphocholine (DSPC) and cholesterol (CHO), the shell defining an inner compartment.

22. The liposomal drug delivery system (LDDS) according to claim 21, wherein the liposomal shell further comprises a surfactant.

23. The liposomal drug delivery system (LDDS) according to claim 22, wherein the surfactant is at least one surfactant selected from the group consisting of: dioctyl sulfosuccinate (DOS), Tween 80 and Span 80, or any combination thereof.

24. The liposomal drug delivery system (LDDS) according to claim 20, further comprising a polymeric coating at least partially covering the shell.

25. The liposomal drug delivery system (LDDS) according to claim 24, wherein the polymeric coating is at least one polymeric coating selected from the group consisting of: biocompatible polymers, ionic polymers, anionic, cationic polymers, gelatin, polyethyleneimine (PEI), poly-L-lysine (PLL), carrageenan, pectin, sodium alginate, carboxylic, sulfate, and amine functionalized polymers such as polyacrylic acid (PAA), polymethacrylic acid, polyethylene amine, polysaccharides such as alginic acid, pectinic acid, carboxy methyl cellulose, hyaluronic acid, heparin (mucopolysaccharide), chitosan, carboxymethyl chitosan, carboxymethyl starch, carboxymethyl dextran, heparin sulfate, chondroitin sulfate, cationic guar, cationic starch, and their salts, poly (butyl cyanoacrylate) (PBCA), poly(lactic acid) (PLA), poly(propylene fumarate)(PPF), polyanhydrides.

26. The liposomal drug delivery system (LDDS) according to claim 25, wherein the polymeric coating comprises at least two layers, preferably an anionic layer and a cationic layer, further preferably polyacrylic acid (PAA) and chitosan (CHT).

27. The liposomal drug delivery system (LDDS) according to claim 21, wherein the nanoliposomal shell further comprises a lyoprotectant, preferably a sugar, further preferably lactose and/or fructose.

28. The liposomal drug delivery system (LDDS) according to claim 21, further comprising a gas housed within the inner compartment defined by the shell so as to form a nanolipobubble (NLB).

29. The liposomal drug delivery system (LDDS) according to claim 28 wherein the gas is at least one gas selected from the group consisting of, air, nitrogen, oxygen, carbon dioxide, hydrogen, nitrous oxide, a noble or inert gas such as helium, argon, xenon or krypton; a radioactive gas such as Xe¹³³ or Kr⁸¹; a hyperpolarized noble gas, a low molecular weight hydrocarbon such as methane, ethane, propane, butane, isobutane, pentane or isopentane; a cycloalkane such as cyclobutane or cyclopentane; an alkene such as propene, butene or isobutene; or an alkyne such as acetylene; an ether; a ketone; an ester; halogenated gases, preferably fluorinated or perfluorinated gases, such as fluorinated hydrocarbons; sulphur hexafluoride; perfluoroacetone; perfluorodiethyl ether; perfluoroalkanes; perfluoroalkenes; perfluoroalkynes; perfluorocycloalkanes; and saturated perfluorocarbons, preferably, the gas is sulphur-hexa fluoride.

30. The liposomal drug delivery system (LDDS) according to claim 21, further comprising a drug compound housed inside the inner compartment defined by the liposomal shell.

31. The liposomal drug delivery system (LDDS) according claim 30, wherein the drug compound is at least one drug selected from the group consisting of: amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, antiparkinson agents, antidepressants, anti-neoplastic drugs, immunosuppressants, antiviral agents, antibiotic agents, antifungal agents, antimicrobial agents, appetite suppressants, antiemetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds; oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol-lowering agents, phytochemicals, and antioxidants, or any derivative of the aforementioned.

32. The liposomal drug delivery system (LDDS) according to claim 31, wherein the anti-neoplastic drug is at least one anti-neoplastic drug selected from the group consisting of: camptothecin, taxanes and platinum compounds.

33. The liposomal drug delivery system (LDDS) according to claim 21, wherein the distearoyl phosphocholine (DSPC) is 1,2-distearoyl-sn-glycero-3-phosphocholine.

34. The liposomal drug delivery system (LDDS) according to claim 21, wherein the liposomal shell is configured such that non-polar functional groups of the distearoyl phosphocholine (DSPC) and the cholesterol (CHO) are directed inwardly toward the inner compartment and polar functional groups are directed outwardly toward an outer surface of the shell, in use, the non-polar functional groups of the liposomal shell increases the solubilisation of non-polar and/or lipophilic drug compounds housed within the compartment.

35. A liposomal drug delivery system (LDDS) for the release of at least one drug compound to a target site in a human or animal body, the liposomal drug delivery system (LDDS) comprising:
a nanoliposomal shell consisting of distearoyl phosphocholine (DSPC), cholesterol (CHO) and a surfactant, the shell defining an inner compartment; and
a drug compound housed inside the inner compartment defined by the nanoliposomal shell.

36. The liposomal drug delivery system (LDDS) according to claim 35, wherein the surfactant is dioctyl sulfosuccinate (DOS).

37. The liposomal drug delivery system (LDDS) according to claim 35, further comprising a polymeric coating at least partially covering the shell, preferably the polymeric coating comprises at least two layers, preferably an anionic layer and a cationic layer, further preferably polyacrylic acid (PAA) and chitosan (CHT).

38. The liposomal drug delivery system (LDDS) according to claim 35, wherein the nanoliposomal shell further comprises a lyoprotectant, preferably a sugar, further preferably lactose and/or fructose.

39. The liposomal drug delivery system (LDDS) according to claim 35, further comprising a gas housed within the inner compartment so as to form a nanolipobubble (NLB).

40-49. (canceled)
